(19) United States
(12) Patent Application Publication
(10) Pub. No.: US 2025/0339567 A1
(43) Pub. Date: Nov. 6, 2025

(54) COMPOUNDS WITH IMPROVED PHARMACOKINETICS FOR IMAGING AND THERAPY OF CANCER

(71) Applicant: Technische Universität München, Munchen (DE)

(72) Inventors: Hans-Jürgen Wester, Schweitenkirchen (DE); Thomas Günther, München (DE)

(21) Appl. No.: 17/768,706

(22) PCT Filed: Oct. 21, 2020
(86) PCT No.: PCT/EP2020/079627

§ 371 Date: Apr. 13, 2022

(30) Foreign Application Priority Data
Dec. 19, 2019 (EP) 19217951.3

(51) Int. Cl. A61K 51/08; A61P 35/00

(52) U.S. Cl. CPC A61K 51/088; A61P 35/00; A61K 2121/00; A61K 2123/00

(57) ABSTRACT

The present invention relates to a compound binding to an endogenous receptor, said compound comprising (i) an oligopeptide comprising a dipeptide with Trp being the C-terminal amino acid of said dipeptide, wherein said Trp is replaced with an α-amino acid Xaa₂, whereby the stability in serum or plasma of the peptide bond connecting Xaa₂ to the N-terminally adjacent amino acid is increased as compared to the peptide bond connecting Trp to the N-terminally adjacent amino acid in an otherwise identical compound; and (ii) a moiety capable of generating therapeutically effective radiation, said moiety being covalently bound to said oligopeptide.

Due to poor survival rates at progressed stages of disease, prostate cancer (PCa), one of the most common malignant diseases in men in the Western world, remains a difficult task to medical treatment. As it is shown that treatment success is higher the sooner it is diagnosed, new methods are warranted. Over the last decades, diagnosis and therapy of cancers by nuclear medicine based on radioactive tracers which accumulate fast and almost exclusively at the tumor site, have gathered growing interest.

Showing several excellent properties as overexpression in prostate cancer as well as low expression in healthy tissues, fast clearance rates and high occurrence (92% of all prostate cancers), prostate-specific membrane antigen (PSMA) tracers are frequently used for endoradiotherapy and imaging of PCa. Nevertheless, there are disadvantages using PSMA such as rather low expression in early states of disease as well as high uptake in the kidneys and salivary glands.

As an interesting alternative, the Gastrin-Releasing Peptide Receptor (GRPR) also shows good occurrence in PCa (up to 100% in early stages, 60% in later stages), is overexpressed in malignant tissue and shows only high expression in one healthy tissue (pancreas). This is an advantage compared to PSMA in case of metastases headed in the area of the kidneys which cannot be detected properly by using PSMA tracers due to the high uptake in the kidneys. Further, a growing concern using high therapeutic doses seems to be the damage of the salivary glands and kidneys due to high accumulation of PSMA tracers.

GRPR is found to show higher expression in early stages of PCa whereas PSMA overexpression is observed the more at later stages of disease. Furthermore, GRPR overexpression is additionally found in estrogen receptor (ER) rich breast cancer which allows the usage of the same tracers for different cancers and genders. Therefore, GRPR tracers are a useful tool as an alternative for patients with low PSMA expression or better diagnosis of metastases located in the kidney area. A contingent therapy of prostate cancers (in early stages) is beneficiary with GRPR tracers instead of PSMA tracers due to higher expression rates and lower side effects (salivary gland damage). Additionally, GRPR antagonists enable the possibility of being used for different genders as it is overexpressed in prostate and breast cancer.

To date, both, GRPR agonists and antagonists have been and are currently used in clinical settings. As agonists show some painful side effects after application to the patients on the one hand and have worse pharmacokinetics due to a by far slower washout from non-tumor tissues, the development of antagonists is increasing. There are noticeably fewer GRPR derivatives in clinical use than PSMA ligands. However, as only 92% of all PCa tumors express PSMA and GRPR is also overexpressed in about 85% of all estrogen receptor (ER) rich breast cancers, there is a clinical benefit.

The general necessary structure of an antagonistic GRPR molecule comprises a binding unit which is based on the native Bombesin or C-terminal part of the Gastrin-releasing peptide (GRP) for its subnanomolar affinity. A linker moiety between the pharmacophoric section and the N-terminal chelator is not necessarily required as there are tracers which show good performance nonetheless but there are also many reports demonstrating beneficiary effects in terms of pharmacokinetics using a linker unit.

Among GRPR antagonists, the derivative RM2 (DOTA-Pip-D-Phe-Gln-Trp-Ala-Val-Gly-His-Sta-Leu-NH₂) is the most often used agent for selective GRPR imaging and therapy. It is predominantly labeled with ⁶⁸Ga (88.9% β⁺, E_{β+, max}=1.89 MeV, t_{1/2}=68 min) for imaging and with ¹⁷⁷Lu (78.6% β⁻, E_{β, max}=0.498 MeV, t_{1/2}=6.7 d) for endoradiotherapy and can be applied to PCa and ER rich breast cancers whereby it is so far considered as the golden standard among GRPR antagonists.

Both, ⁶⁸Ga- and ¹⁷⁷Lu-RM2 show favorable pharmacokinetics as high tumor accumulation, fast clearance from non-tumor tissue and good retention in the tumor over a large time span in humans leading to a high contrast and good therapy results, respectively.

Nonetheless, certain Bombesin analogs are metabolically unstable in animals which limits the desired accumulation in tumor tissues.

On the other hand, it has to be mentioned that more stable GRPR derivatives show slower washout from the GRPR rich pancreas which has to be considered before the usage for therapy of humans due to a possible pancreatitis.

In other malignant indications, yet further markers and targets are of interest. These include Neuromedin-B receptor (Bombesin-1 receptor, NMBR), Bombesin receptor subtype 3 (BRS-3) and Cholecystokinin-2 receptor (CCK-2R).

In view of the above, the technical problem underlying the present invention can be seen in the provision of improved radiopharmaceuticals and radiodiagnostics, in particular in the field of cancer, improvements including pharmacokinetic properties.

This technical problem has been solved by the subject-matter disclosed below.

In a first aspect, the present invention relates to a compound binding to an endogenous receptor, said compound comprising (i) an oligopeptide comprising a dipeptide with Trp being the C-terminal amino acid of said dipeptide, wherein said Trp is replaced with an α-amino acid Xaa₂, whereby the stability in serum or plasma (preferably mammalian serum or plasma) of the peptide bond connecting Xaa₂ to the N-terminally adjacent amino acid is increased as compared to the peptide bond connecting Trp to the N-terminally adjacent amino acid in an otherwise identical compound; and (ii) a moiety capable of generating therapeutically effective radiation, said moiety being covalently bound to said oligopeptide.

A receptor is a molecule capable of specifically binding its cognate ligand. The term “cognate ligand” designates a genus of molecules and embraces the natural ligand and the compounds in accordance with the invention. The receptor is preferably a polypeptide or protein. It may comprise a plurality of subunits which may be non-covalently or covalently connected to each other. Preferably said receptor is a transmembrane protein or a membrane-associated protein. Preferably, the ligand-binding site is located extracellularly.

The term “endogenous” refers to the occurrence of the receptor in the human or animal body, animals including mammals, mammals including rodents. Preferred receptors are the subject of a preferred embodiment disclosed further below.

The compound of the first aspect comprises or consists of two moieties. The first moiety is a targeting moiety. It comprises or consists of the above disclosed oligopeptide. The second moiety is a moiety which conveys the intended therapeutic effect which is, in case of the compound in accordance with the first aspect, radiation. As such, it is understood that therapy involves the destruction of the targeted tissue, in general because the targeted tissue is or comprises hyperproliferative tissue such as malignant tissue.

As will become apparent further below, in other aspects of the invention the second moiety serves diagnostic purposes.

In its broadest definition, the second moiety is not particularly limited other than that it has to be capable of generating therapeutically effecting radiation. In accordance with the invention, this capability is conveyed by radionuclides. Such radionuclide may be present in said compound or, in the alternative, the compound may be equipped with a moiety, said moiety in turn being capable of being loaded with a radionuclide.

The term “oligopeptide” has its art-established meaning. It is a linear sequence of amino acids which are connected to each other by main chain peptide bonds. In terms of length, 5 to 20 amino acids are preferred. This includes oligopeptides having 6, 7, 8, 9, 10, 11, 12, 13, 14, 15, 16, 17, 18 or 19 amino acids. Preferred are 6, 7, 8, 9 or 10 amino acids. Especially preferred are 9 or 10 amino acids and most preferably 9 amino acids. While the term “oligopeptide” implies a peptidic nature, the term also embraces compounds which are not exclusively predominantly peptidic in nature. Preferably, and assuming that said oligopeptide has N amino acids, at least (N-1)/2 bonds connecting said amino acids are peptide bonds. For example, N-1, N-2 or N-3 bonds connecting said amino acids are peptide bonds.

The same considerations apply mutatis mutandis to the building blocks of the oligopeptide. In other words, at least N/2 building blocks are amino acids. For example, N, N-1, N-2 or N-3 building blocks are amino acids.

The term “amino acid” designates a molecule with a carboxyl and an amino group. Preferred amino acids are α-amino acids including proteinogenic amino acids, but other amino acids such as β-, γ- or δ-amino acids may also be used. In particular in the C-terminal region of the molecule a γ-amino acid may be employed; see preferred embodiments further below.

Overall, preference is given to naturally occurring, preferably proteinogenic α-amino acids. Having said that, for the purpose of conferring specific technical effects as detailed further below, one or more positions, generally not more than a half of the positions of the oligonucleotide, are amino acids or moieties which are not naturally occurring. These are also referred to as modified amino acids or modified moieties herein. Such modifications may affect stereochemistry, for example the use of a D-amino acid instead of its naturally occurring L-counterpart and/or modifications concerning constitution and composition.

To the extent the amino acids are not located at the termini of the molecule, it is understood that a given amino acid is connected to the adjacent moieties via main chain peptide bonds, the consequence being that in such a case there are no free carboxylates and primary amines.

Within said oligopeptide, of particular relevance is a dipeptide unit. The location of the dipeptide unit within said oligopeptide is not particularly limited. Preference is given, though, to said dipeptide unit being located within the N-terminal half of the oligopeptide.

Within said dipeptide, the C-terminal amino acid is a tryptophan derivative. In many instances, the naturally occurring ligand of the mentioned endogenous receptor is also peptidic in nature and has a tryptophan at the corresponding position. A corresponding position is a position which aligns in a sequence alignment of the naturally occurring ligand with a compound of the first aspect.

In accordance with the invention, such tryptophan is to be modified. As will become apparent further below, preferred modifications are those which maintain the indole ring. Furthermore, the amino and carboxy functionalities are retained. In that sense, the meaning of the term “derivative” is accordingly limited: the derivative has to be an aromatic amino acid, preferably with a two-membered ring, more preferably an indole ring. Moreover, in accordance with the invention, the tryptophan derivative is an α-amino acid.

In accordance with the invention, the modification of tryptophan serves to increase the stability in serum or plasma of the peptide bond connecting the tryptophan derivative (also referred to as Xaa₂) with the N-terminally adjacent amino acid.

The terms “increase of stability in serum or plasma of a peptide bond” and “decrease of cleavage in serum or plasma of a peptide bond” are used equivalently herein.

Stability in serum or plasma is preferably in mammalian serum or plasma. Particularly preferred, and in view of the preferred applications, stability in serum or plasma is stability in human serum or plasma. For the purpose of testing and development, a preferred serum or plasma is rodent serum or plasma such as murine serum or plasma.

For determining stability in serum or plasma, a compound of the invention is e.g. incubated at 37° C. for 3 days (e.g. 72±2 h).

Assays for determining stability in serum or plasma are well-established in the art and include in vitro and in vivo assays. Exemplary or preferred assays are part of the examples enclosed herewith. For the purpose of determining whether stability is increased, use is made a reference compound. The reference compound for assessing compounds of the first aspect is chosen such that the only difference between the compound under consideration and the reference compound is the position Xaa₂. In the reference compound, said position is tryptophan.

It is understood that increased stability means statistically significantly increased stability and/or at least 1.1-fold, at least 1.2-fold, at least 1.3-fold, at least 1.4-fold, at least 1.5-fold, at least 2-fold, at least 3-fold, at least 4-fold, at least 5-fold, at least 10-fold, at least 50-fold, or at least 100-fold increased stability. A preferred parameter for determining the mentioned increase is serum/plasma half-life. A preferred parameter for determining the mentioned increase is the amount of intact radiolabeled compound after incubation in human/murine serum or plasma for 72±2 h.

In an alternative approach, the respective cognate ligand of said endogenous receptor or an established therapeutic agent binding to the same receptor (such as RM2 in case of GRPR being the receptor, see also below) may be used as a reference compound.

Compounds in accordance with the first aspect show enhanced pharmacokinetic properties. The reference compound for comparison is specified above and is a compound which deviates from the compound in accordance with the first aspect under consideration only in that at the position where the compound in accordance with the first aspect has Trp derivative, an unmodified tryptophan is present in said reference compound. Alternatively, enhancement is as compared to the respective natural ligand and/or an art-established therapeutic agent targeting the same receptor. To the extent compounds of the first aspect are considered which are GRPR ligands, a preferred art-established compound is RM2 (DOTA-Pip-D-Phe-Gln-Trp-Ala-Val-Gly-His-Sta-Leu-NH₂, wherein the abbreviations of the chelating agent as of well as non-proteinogenic amino acids are explained further below).

Natural ligands of preferred receptors in accordance with the invention, said preferred receptors being the subject-matter of a preferred embodiment disclosed further below, are as follows: neuromedine-B in case of the neuromedine-B receptor, gastrin-releasing peptide in case of the gastrin-releasing peptide receptor, and Gastrin in case of the cholecystokinin-2 receptor.

In the context of therapy, it is understood that high tumor uptake and/or tumor retention are desirable. Evidence in that respect is given in the examples enclosed herewith.

The technical means to achieve high tumor uptake and retention are given above: it is the stabilization of the peptide bond within the dipeptide unit comprised in compounds in accordance with the first aspect.

In a preferred embodiment of the compound of the first aspect, said N-terminally adjacent amino acid in said dipeptide is L-Gln, D-Gln, L-His, D-His or Gly, preferably L-Gln.

In a further preferred embodiment, said endogenous receptor is a peptide receptor overexpressed in cancer disease, such as Neuromedin-B receptor (Bombesin-1 receptor, NMBR), Gastrin-releasing peptide receptor (Bombesin-2 receptor, GRPR), Bombesin receptor subtype 3 (BRS-3) or Cholecystokinin-2 receptor (CCK-2R), and wherein preferably (a) said binding is with a K_{D} of less or equal 50 nM, less or equal 15 nM, less or equal 5 nM or less or equal 1 nM; and/or (b) said compound is a GRPR antagonist, preferably with an IC₅₀ of less or equal 50 nM, less or equal 15 nM, less or equal 5 nM or less or equal 1 nM.

In a second aspect, which is related to the first aspect, the present invention provides a compound of formula (I)

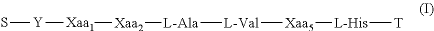

wherein
S is a moiety capable of generating therapeutically active radiation;
Y is an optional linker;
Xaa₁ is (i) L-Gln, D-Gln, L-His, D-His or Gly, preferably L-Gln; or (ii) an α-amino acid which increases stability in serum or plasma of the Xaa₁-Xaa₂ peptide bond as compared to Xaa₁ being Gln and Xaa₂ being Trp in an otherwise identical compound;
Xaa₂ is Trp or an α-amino acid which increases stability in serum or plasma of the Xaa₁-Xaa₂ peptide bond as compared to Xaa₁ being Gln and Xaa₂ being Trp in an otherwise identical compound;
provided that Xaa₁ is not any one of L-Gln, D-Gln, L-His, D-His and Gly, and Xaa₂ is not Trp, respectively, at the same time;
Xaa₅ is Gly, N-Me-Gly, D-Ala, β-Ala or 2-aminoisobutyric acid (Aib); preferably Gly; and
T is an optional terminal group.

The compound of the second aspect is tailored to the specific endogenous receptor which is GRPR. As such, it comprises several features which are inherited from the natural cognate ligand which is gastrin releasing peptide (GRP).

In accordance with the second aspect, the moiety capable of generating therapeutically active radiation is located at the N-terminus. The core of the compound of the second aspect is an oligopeptide with 6 amino acids, wherein the dipeptide defining the peptide bond to be stabilized in accordance with this invention is located at positions 1 and 2 of the core oligopeptide.

The optional linker Y may be present or not, and, to the extent present, it may be a means to incorporate further amino acids into the compound of the second aspect.

Also the optional terminal group T may be, but does not have to be, a means to extend the peptidic part of the compound of the second aspect.

The reference compound for determining whether stability in serum or plasma is increased is a compound which deviates from the compound of Formula (I) under consideration in that Xaa₁ is Gln and Xaa₂ to Trp. As noted above in relation to the compound of the first aspect, alternative reference compounds may be employed, which alternative reference compounds include the natural ligand and art-established pharmaceuticals binding to GRPR such as RM2.

In a preferred embodiment of the compounds of the first and second aspect, Xaa₂ is (a) Trp which is modified to comprise (i) a C1 to C4 optionally substituted alkyl moiety bound to the α-carbon, substituents being selected from halogen and hydroxyl; and/or (ii) a substituent bound to the indole ring, substituents being selected from N-(2,2,2-trifluoromethyl), N-methyl, N-acetyl, 5-fluoro, 5-bromo, 5-iodo, 5-chloro, 5-hydroxy, 5-methoxy, 5-methyl, 6-chloro, 7-chloro and 7-Aza; (b) 1,2,3,4-tetrahydro norharmane-3-carboxylic acid (L-Tpi).

This preferred embodiment relates to specific structural means of achieving increased stability of the main chain peptide bond of the dipeptide moiety present in the compounds of the first and second aspect (designated Xaa₁-Xaa₂ in case of the compound of the second aspect).

Particularly preferred among these structural measures are those specified in part (a) (i) of this preferred embodiment.

More preferred implementations thereof are such that said optionally substituted alkyl moiety is selected from —CH₃, —CH₂CH₃, and CHₙHal₃₋ₙ, wherein n is 0, 1 or 2 and Hal is F, Cl, Br and/or I such as —CF₃; and preferably is —CH₃.

Most preferred is that Xaa₂ is α-methyl tryptophan.

Preferred embodiments of the first and second aspect are derivatives of the compounds of Table 1A and/or B. Table 1A and B is presented further below as is an explanation of the term “derivative” of compounds of Table 1A and B.

A particularly preferred embodiment of the compounds of the first and second aspect is the compound of Formula (IIIa and IIIb) as disclosed further below.

A third aspect of the invention relates to a compound of formula (II)

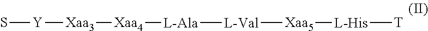

wherein
S is a moiety capable of generating a detectable signal;
Y is an optional linker;
Xaa₃ is (i) L-Gln, D-Gln, L-His, D-His or Gly, preferably L-Gln; or (ii) an α-amino acid which decreases stability in serum or plasma of the Xaa₃-Xaa₄ peptide bond as compared Xaa₃ being Gln and Xaa₄ being Trp in an otherwise identical compound;
Xaa₄ is Trp or an α-amino acid which decreases stability in serum or plasma of the Xaa₃-Xaa₄ peptide bond as compared Xaa₃ being Gln and Xaa₄ being Trp in an otherwise identical compound;
wherein said α-amino acid at position Xaa₄ which decreases stability in serum or plasma of the Xaa₃-Xaa₄ peptide bond is not a proteinogenic amino acid;
provided that Xaa₃ is not any one of L-Gln, D-Gln, L-His, D-His and Gly, and Xaa₄ is not Trp, respectively, at the same time;
Xaa₅ is Gly, N-Me-Gly, β-Ala or 2-aminoisobutyric acid (Aib); preferably Gly; and T is an optional terminal group.

While exhibiting structural similarity to the compound of Formula (I) in accordance with the second aspect, the compounds of Formula (II) are distinct in that the peptide bond in the dipeptide moiety comprised in the oligopeptide has less stability in serum or plasma.

This provides for a distinct but related technical effect: as is well-established in the art, radiolabeled compounds are useful not only for therapeutic, but also for diagnostic purposes. In a diagnostic setting, more rapid degradation is desirable. This is because metabolic activity in a tumor is generally lower than in the surrounding normal tissue, the consequence being that more rapid degradation entails a higher tumor-to-background ratio, such higher tumor-to-background ratio allowing for more sensitive, more precise and/or more accurate detection of tumors and metastases.

It is understood that the two positions Xaa₃ and Xaa₄ correspond and align with positions Xaa₁ and Xaa₂ of the compounds of the second aspect and are merely labeled distinctly for the sake of clarity. When it comes to the concrete structurally implementation, Xaa₁ and Xaa₂ on the one hand and Xaa₃ and Xaa₄ on the other hand will generally be distinct. This will become more apparent in the context of preferred embodiments of the third aspect as disclosed further below.

For the purpose of determining decreased stability, the explanations given above in relation to compounds of the first and second aspect apply mutatis mutandis. Accordingly, in vitro and in vivo serum or plasma assays may be used. A preferred readout is the serum/plasma half-life. A more preferred readout is the amount of intact radiolabeled compound after incubation in human/murine plasma for 72±2 h. Reference compounds for the purpose of determining decreased stability include, as recited above, a compound which differs from the compound of Formula (II) only in that positions Xaa₃ and Xaa₄ are Gln and Trp, respectively.

As established in the art, the three letter code is generally used for designating amino acids. If the first letter is an upper case letter, the L-form is intended, whereas if the first letter is lower case letter, the D-form is intended. To give an example, Trp refers to L-tryptophan, whereas trp designates D-tryptophan. Also the explicit indication of the stereochemistry is used herein (such as L-Trp and D-Trp).

Alternative reference compounds are the respective natural ligand which is GRP in case of the receptor being GRPR, or RM2 (which is antagonistic).

In a preferred embodiment of the compounds of Formula (II), Xaa₃ is Hse and/or Xaa₄ is Bta (3-benzothienyl alanine).

In a preferred embodiment of the compounds of the second aspect, S is selected from a radioactive moiety and a moiety capable of being loaded with a radioactive nuclide.

In a preferred embodiment of the compounds of the third aspect, S is selected from a fluorescent moiety, a radioactive moiety, and a moiety capable of being loaded with a radioactive nuclide.

The preceding two preferred embodiments relate to preferred implementations of the moiety S depending on whether a therapeutic or diagnostic compound is under consideration.

To the extent use is made of a moiety which is capable of being loaded with a radioactive nuclide, said moiety preferably is a metal ion chelator, preferably selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]hexadecane (DO2A), 1,4,7,10-tetraazacyclododecane-N,N′,N″,N′″-tetraacetic acid (DOTA), α-(2-carboxyethyl)-1,4,7,10-tetraazacyclododecane-1,4,7,10-tetraacetic acid or 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglycol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecane-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridin-bis(methyleneamintetraacetic acid (TMT), 1,4,7,10-tetraazacyclotridecan-N,N′,N″,N′″-tetraacetic acid (TRITA), triethylenetetraaminehexaacetic acid (TTHA), N,N′-bis[(6-carboxy-2-pyridyl)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 6-carboxy-1,4,8,11-tetraazaundecane (N4), 6-{p-[(carboxymethoxy)acetyl]-aminobenzyl}-1,4,8,11-tetraazaundecane (N4′), 1,4,7,10-Tetraazacyclododecane-1,4,7-triacetic acid (DO3A), S-acetylmercaptoacetyltriserine (MAS3), Mercaptoacetyltriglycine (MAG3), 1,4-bis (hydroxycarbonyl methyl)-6-[bis(hydroxylcarbonyl methyl)] amino-6-methyl perhydro-1,4-diazepine (AAZTA), 3,6,9,15-tetraazabicyclo[9.3.1]pentadeca-1 (15), 11,13-triene-2,10-dione (TBPD), 9-oxa-3,6,12,15,21-pentaazatricyclo[15,3,2,1] trieicos-1 (21), 17,19-triene-2,7,11,16-tetradione (OPTT), 2-[Bis(carboxymethyl)aminomethyl]-2-[(4-isothiocyanatobenzyl)oxy-methyl]propylene-1,3-dinitrilotetraacetic Acid (TAME-Hex), 4-((4-(3-(bis(2-(3-hydroxy-1-methyl-2-oxo-1,2-dihydropyridine-4-carboxamido)ethyl)amino)-2-((bis(2-(3-hydroxy-1-methyl-2-oxo-1,2-dihydropyridine-4-carboxamido)ethyl)amino)methyl)propyl)phenyl)amino)-4-oxobutanoic acid (Me-3,2-HOPO), 2,20-(6-((carboxymethyl)amino)-1,4-diazepane-1,4-diyl)diacetic acid) (DATA), 1,4,7-triazacyclononane-1,4,7-tris[methyl (2-carboxyethyl)phosphinic acid] (TRAP) and functional derivatives thereof, such as NOPO (1,4,7-triazacyclononane-1,4-bis[methylene(hydroxymethyl)phosphinic acid]-7-[methylene(2-carboxyethyl)phosphinic acid]), 1,4,7,10-tetraazacyclododecane-1,4,7,10-tetrakis[methylene(2-carboxyethyl)phosphinic acid] (DOTPI), 6,6′-({9-hydroxy-1,5-bis(methoxycarbonyl)-2,4-di(pyridin-2-yl)-3,7-diazabicyclo[3.3.1]nonane-3,7-diyl}bis(methylene))dipicolinic acid (H2bispa2), 1,4,7,10,13-pentaazacyclopentadecane-N,N′,N″,N″,N′″-pentaacetic acid (PEPA), 1,4,7,10,13,16-hexaazacyclohexadecane-N,N′,N″,N′″,N″″,N′″″-hexaacetic acid (HEHA), 1,2-[{6-(carboxy)-pyridin-2-yl}-methylamino]ethane (H₂dedpa), N,N′-bis{6-carboxy-2-pyridylmethyl}-ethylenediamine-N,N′-diacetic acid (H₄octapa), 4,10-bis(carboxymethyl)-1,4,7,10-tetraazabicyclo[5.5.2]tetradecane (CB-DO2A), 1,4,7,10-tetrakis(carbamoylmethyl)-1,4,7,10-tetraazacyclododecane (TCMC), 1,8-diamino-3,6,10,13,16,19-hexaazabicyclo[6.6.6]icosane (sar) and functional derivatives thereof, {4-[2-(bis-carboxymethylamino)-ethyl]-7-carboxymethyl-[1,4,7]triazonan-1-yl}-acetic acid (NETA), N,N′,N″, tris(2-mercaptoethyl) 1,4,7-triazacyclononane (TACN-TM), 2-(p-isothiocyanatobenzyl)-cyclohexyldiethylenetriaminepentaacetic acid (CHX-A″-DTPA), N,N′-[1-benzyl-1,2,3-triazole-4-yl]methyl-N,N′-[6-(carboxy)pyridin-2-yl]-1,2-diaminoethane (H₂azapa), N,N″-[[6-(carboxy)pyridin-2-yl]methyl]diethylenetriamine-N,N′,N″-triacetic acid (H₅decapa), N,N′-bis(2-hydroxy-5-sulfobenzyl)ethylenediamine-N,N′-diacetic acid (SHBED), 3,6,9,15-tetraazabicyclo[9.3.1]pentadeca-1 (15), 11,13-triene-3,6,9,-triacetic acid (PCTA), and N,N-(methylenephosphonate)-N,N′-[6-(methoxycarbonyl)pyridin-2-yl]methyl-1,2-diaminoethane (H₆phospa), more preferably being DOTA or DOTAGA; wherein preferably a radioactive cation is bound to said chelator, said radioactive cation preferably being selected from ⁴³Sc, ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴⁰La, ¹⁴²La, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁶Dy, ¹⁶⁵Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ¹⁸⁸Re, ¹⁸⁸W, ¹⁹¹ Pt, ^{195m}Pt, ¹⁹⁴ Ir, ¹⁹⁷Hg, ¹⁹⁸Au, ¹⁹⁹ Au, ²¹²Pb, ²⁰³Pb, ²¹¹ At, ²¹²Bi, ²¹³Bi, ²²³Ra, ²²⁴Ra, ²²⁵Ac, and ²²⁷Th, or a cationic molecule comprising ¹⁸F, such as ¹⁸F-[AIF]²⁺.

For therapeutic compounds, a preferred nuclide is ¹⁷⁷Lu. An example of a preferred nuclide for diagnostic compounds is ⁶⁸Ga.

In a preferred embodiment of the compounds of the second and third aspect, the linker Y is present and (a) comprises one, two, three, four, five or six positive and/or negative charge(s); (b) comprises or consists of one, two, three, four, five or six amino acids, preferably (a) D-amino acid(s) being among said amino acids, more preferably (a) D-α-amino acid(s); (c) comprises or consists of PEGₙ, n being an integer selected from 1, 2, 3, 4, 5, 6, 7, 8, 9 and 10; and/or (d) comprises a moiety capable of generating a detectable signal.

Suitable moieties capable of generating a detectable signal in accordance with item (d) of the above preferred embodiment may be moieties which are fluorescent or moieties comprising or capable of being loaded with a radionuclide. An example of the latter is a silicon fluoride acceptor moiety (SiFA) which can be used for ¹⁸F labeling. To the extent compounds of the invention which comprise such a SiFA moiety furthermore comprise a chelating agent (such as DOTA or DOTAGA), such a compound would comprise two radionuclides and could therefore be used for both diagnosis and therapy.

In a preferred embodiment, said SiFA moiety has a structure represented by formula (VI).

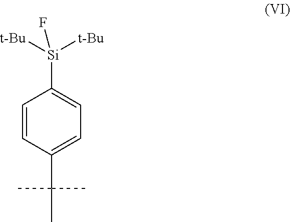

wherein
t-Bu indicates a tert-butyl group; and
the dashed line marks the bond which attaches the moiety to the remainder of the compound.

Preferred attachment sites of the SiFA moiety within the linker Y is the side chain of 2,3-diaminopropionic acid, said side chain consisting of —CH₂—NH₂, wherein the terminal amino group of said side chain preferably forms an amide bond with a carboxyl group bound to the free valence of the SiFA moiety in formula (VI).

Linkers Y with a silicon fluoride acceptor moiety are preferred linkers Y of compounds of all aspects of the present invention.

In a further preferred embodiment said linker Y comprises or consists of (a) D-Glu-urea-D-Glu; (b) one or two 2,3-diaminopropionic acid moieties, optionally substituted with a moiety capable of generating a detectable signal; (c) one, two, three, four, five or six consecutive amino acids comprising or consisting of one or more amino acids selected from D-/L-aspartate, D-/L-ornithine, 4-amino-1-carboxymethyl-piperidine (Pip), D-/L-2,3-diaminopropionic acid, D-/L-serine, D-/L-citrulline moieties, L-cysteic acid (Ala(SO₃H)), amino-valeric acid (Ava), 4-aminobenzoic acid (PABA) and D-Phe; and/or; (d) p-aminomethylaniline-diglycolic acid (abbreviated pABza-DIG or AMA-DGA, and/or diglycolate (abbreviated DIG or DGA).

Particularly preferred is that Y is Pip-phe.

The D-Glu-urea-D-Glu moiety in accordance with item (a) of this preferred embodiment is considered to be a means of rendering the compound more hydrophilic.

In a further preferred embodiment of compounds of the second and third aspect, i.e. of both therapeutically active and diagnostically active agents, the terminal group T is present and comprises or consists of (a) statine (Sta or (3S,4S)-4-amino-3-hydroxy-6-methylheptanoic acid), 2,6-dimethyl heptane, Leu or β-thienyl-L-alanine (Thi); and/or (b) Leu, norleucine (Nle), Pro, Met, or 1-amino-1-isobutyl-3-methyl-butane, wherein the amidic amine group of said Leu may be modified with ethyl (NH-Ethyl) or NH₂ (NH—NH₂); and/or (c) (S)-1-((S)-2-amino-4-methylpentyl) pyrrolidine-2-carboxamide (Leu-ψ(CH₂N)-Pro-NH₂); provided that if T is or terminates with an amino acid, the carboxylate of said amino acid is amidated.

Particularly preferred is that T is Sta-Leu-NH₂.

As regards preferred choices of moieties Y and T, generally no distinction is made between therapeutic and diagnostic compounds of the invention.

As already noted further above, in a preferred embodiment of the compounds of all aspects of the present invention, said serum or plasma is human serum or plasma. In other words, what particularly matters is increased or decreased stability in human serum or plasma, respectively.

Table 1A below displays the sequences of known GRPR-binding agents. The hexapeptide sequences beginning with Xaa₁ and ending with L-His in case of the compound of formula (I), and beginning with Xaa₃ and ending with L-His in case of the compound of formula (II) corresponds to positions 7 to 12 in the table below. As can be recognized, the GRPR-binding agents shown in this table throughout have tryptophan at position 8 (which corresponds to position Xaa₂ and Xaa₄, respectively). Position 7 (corresponding to Xaa₁ and Xaa₃, respectively) is highly conserved. It is apparent from the table below that in the art there is no recognition of the peptide bond connecting positions 7 and 8 (numbering as in the table) being a target site for fine-tuning pharmacokinetic properties.

Table 1B and 1C display the sequences of modified GRPR-addressing ligands as well as the effects of the introduction of either a α-Me-Trp or Bta moiety at position 8 or a Hse moiety at position 7 into different GRPR-targeted compounds. Similarly to Table 1A, the hexapeptide sequences beginning with Xaa₁ and ending with L-His in case of the compound of formula (I), and beginning with Xaa₃ and ending with L-His in case of the compound of formula (II) corresponds to positions 7 to 12.

Problems occurring from the metabolic degradation of linear GRPR-targeted peptides are suggested to be caused by the neutral endopeptidase (NEP, EC 3.4.24.11), which is known to cleave linear peptides at the N-terminal side of hydrophobic amino acids (e.g. tryptophan). It is therefore assumed that these peptides are cleaved at the dipeptidic Gln⁷-Trp⁸ motif present in almost every GRPR-addressing compound (Table 1A). In order to prove an increased metabolic stability in human serum or plasma when introducing α-Me-Trp or Hse at the described positions, different GRPR-targeted ligands were synthesized and the above-mentioned modifications were introduced. For almost all evaluated GRPR-targeted peptides shown in Table 1B, a substitution of Trp⁸ by α-Me-Trp⁸ or Gln⁷ by Hse⁷ led to enhanced metabolic stabilities compared to the respective Gln⁷-Trp⁸ comprising derivatives (Table 1C). For most of these analogs, GRPR affinity was not drastically reduced by the addition of α-Me-Trp. However, a substitution of Gln⁷ by Hse⁷ led to a distinct decrease in GRPR affinity for most ligands.

Nevertheless, a stabilizing effect of the Hse moiety could be observed.

Similarly, Bta was introduced at the described position in order to prove a decreased metabolic stability in human serum or plasma. For most of the evaluated GRPR-addressing compounds shown in Table 1B, a substitution of Trp⁸ by Bta⁸ indeed led to decreased metabolic stabilities compared to the respective Gln⁷-Trp⁸ comprising derivatives (Table 1C). GRPR affinity was not drastically affected by the addition of Bta for most derivatives shown in Table 1B.

It is therefore concluded that, concerning GRPR-targeted ligands, it is not crucial, which amino acids are located at the N- and C-terminal side of the Gln⁷-Trp⁸ dipeptide. In general, the introduction of Bta⁸ decreases, whereas the introduction of α-Me-Trp⁸ or Hse⁷ increases metabolic stability in human serum or plasma. Thus, these modifications (α-Me-Trp⁸, Bta⁸ and Hse⁷) enable a broad application throughout GRPR-targeted compounds.

TABLE 1A

| | | 1 | 2 | 3 | 4 | 5 | 6 | 7 |
|---|---|---|---|---|---|---|---|---|
| Bombesin | | pGlu | Gln | Arg | Leu | Gly | Asn | Gln |
| GRP(14-27) | | H-Met | Tyr | Pro | Arg | Gly | Asn | His |
| RM2 (=DOTA-MJ9) | | | | | DOTA | Pip | D-Phe | Gln |
| NeoBOMB1 | | | | DOTA | pABzA | DIG | D-Phe | Gln |
| ProBOMB1 | | | | DOTA | pABzA | DIG | D-Phe | Gln |
| BAY 864367 | | 3-cyano-4-fluoro-benzoyl | | | Ala(SO3H) | Ala(SO3H) | Ava | Gln |
| SB9 | | | | | DOTA | Gly | PABA | Gln |
| SB3 | | | | DOTA | pABzA | DIG | D-Phe | Gln |
| DOTA-(β-Ala)₁₋₃-JMV594 | | | | | DOTA | (β-Ala)₁₋₃ | D-Phe | Gln |
| N4-AR | | | | | N4 | PEG4 | D-Phe | Gln |
| DOTA-AR | | | | | DOTA | PEG4 | D-Phe | Gln |
| NODAGA-AR | | | | | NODAGA | PEG4 | D-Phe | Gln |
| CB-TW2A-AR | | | | | CBTE2a | PEG4 | D-Phe | Gln |
| Demobesin4 | N4 | Pro | Gln | Arg | Tyr | Gly | Asn | Gln |
| AMBA | | | | | DOTA | Gly | PABA | Gln |
| DOTA-PESIN | | | | | | DOTA | dPEG4 | Gln |
| BZH3 | | | | | DOTA | dPEG4 | tyr | Gln |
| 1 | | | | | N4′ | diglycolate | D-Phe | Gln |
| 2 | | | | | N4′ | diglycolate | D-Phe | Gln |
| 3 | | | | | | N4 | D-Phe | Gln |
| 4 | | | | | N4 | PEG2 | D-Phe | Gln |
| 5 | | | | | N4 | AMA-DGA | D-Phe | Gln |
| 6 | | | | | N4 | AMA-DGA | D-Phe | Gln |
| 7 | | | | | N4 | AMA-DGA | D-Phe | gln |
| 8 | | | | | N4 | AMA-DGA | D-Phe | Gln |

| | 8 | 9 | 10 | 11 | 12 | 13 | 14 |
|---|---|---|---|---|---|---|---|
| Bombesin | Trp | Ala | Val | Gly | His | Leu | Met-NH2 |
| GRP(14-27) | Trp | Ala | Val | Gly | His | Leu | Met-NH2 |
| RM2 (=DOTA-MJ9) | Trp | Ala | Val | Gly | His | Sta | Leu-NH2 |
| NeoBOMB1 | Trp | Ala | Val | Gly | His | 2,6-dimethylheptane | |
| ProBOMB1 | Trp | Ala | Val | Gly | His | Leu-ψ(CH2N) | Pro-NH2 |
| BAY 864367 | Trp | Ala | Val | N-Me-Gly | His | Sta | Leu-NH2 |
| SB9 | Trp | Ala | Val | Gly | His | Leu-NH-Ethyl | |
| SB3 | Trp | Ala | Val | Gly | His | Leu-NH-Ethyl | |
| DOTA-(β-Ala)₁₋₃-JMV594 | Trp | Ala | Val | Gly | His | Sta | Leu-NH2 |
| N4-AR | Trp | Ala | Val | Gly | His | Sta | Leu-NH2 |
| DOTA-AR | Trp | Ala | Val | Gly | His | Sta | Leu-NH2 |
| NODAGA-AR | Trp | Ala | Val | Gly | His | Sta | Leu-NH2 |
| CB-TW2A-AR | Trp | Ala | Val | Gly | His | Sta | Leu-NH2 |
| Demobesin4 | Trp | Ala | Val | Gly | His | Leu | Nle-NH2 |
| AMBA | Trp | Ala | Val | Gly | His | Leu | Met-NH2 |
| DOTA-PESIN | Trp | Ala | Val | Gly | His | Leu | Met-NH2 |
| BZH3 | Trp | Ala | Val | β-Ala | His | Thi | Met-NH2 |
| 1 | Trp | Ala | Val | Gly | His | Leu-NH-Ethyl | |
| 2 | Trp | Ala | Val | Gly | His | Leu-NH-NH2 | |
| 3 | Trp | Ala | Val | Gly | His | Leu-NH-Ethyl | |
| 4 | Trp | Ala | Val | Gly | His | Leu-NH-Ethyl | |
| 5 | Trp | Ala | Val | Gly | His | Leu-NH-Ethyl | |
| 6 | Trp | Ala | Val | ala | His | Leu-NH-Ethyl | |
| 7 | Trp | Ala | Val | ala | His | Leu-NH-Ethyl | |
| 8 | Trp | Ala | Val | Gly | His | Leu-NH-NH2 | |

Abbreviations used: Ala(SO₃H) (L-cysteic acid); Ava (amino-valeric acid); CBTE2a (bis(carboxymethyl)-1,4,8,11-tetraazabicyclo[6.6.2]hexadecane); DIG (diglycolic acid); DOTA (1,4,7,10-tetraazacyclododecane-N,N′,N″,N″′-tetraacetic acid); Leu-ψ(CH₂N) (2-amino-4-methylpentane); N4 (6-(carboxy))-1,4,4,11-tetraazaundecane); N4′ (6-{ p-[(carboxymethoxy)acetyl]-aminobenzyl}-1,4,8,11-tetraazaundecane); NODAGA (1-(1-carboxy-3-carboxypropyl)-4,7-(carbooxy)-1,4,7-triazacyclononane); PABA(4-amino benzoic acid); pABZA (p-amino methylaniline); pGlu (pyroglutamic acid); Pip (4-amino-1-carboxymethyl-piperidine); Sta (3S,4S)-4-amino-3-hydroxy-6-methylheptanoic acid); Thi (β-thienyl-L-alanine).

TABLE 1B

| Ligands | 14 | 13 | 12 | 11 | 10 | 9 | 8 | 7 | 6 |
|---|---|---|---|---|---|---|---|---|---|
| [Lu]RM2 | Leu | Sta | His | Gly | Val | Ala | Trp | Gln | phe |
| [Lu]AMTG | Leu | Sta | His | Gly | Val | Ala | α-Me-Trp | Gln | phe |
| [Lu]DOTA-[Bta8]MJ9 | Leu | Sta | His | Gly | Val | Ala | Bta | Gln | phe |
| [Lu]DOTA-[Hse7]MJ9 | Leu | Sta | His | Gly | Val | Ala | Trp | Hse | phe |
| [Lu]DOTA-[N-Me-Gly11]MJ9 | Leu | Sta | His | N-Me-Gly | Val | Ala | Trp | Gln | phe |
| [Lu]DOTA-[α-Me-Trp8, N-Me-Gly11]MJ9 | Leu | Sta | His | N-Me-Gly | Val | Ala | α-Me-Trp | Gln | phe |
| [Lu]DOTA-[Bta8, N-Me-Gly11]MJ9 | Leu | Sta | His | N-Me-Gly | Val | Ala | Bta | Gln | phe |
| [Lu]DOTA-[Hse7, N-Me-Gly11]MJ9 | Leu | Sta | His | N-Me-Gly | Val | Ala | Trp | Hse | phe |
| [Lu]UL | Nle | Phe | His | β-Ala | Val | Ala | Trp | Gln | phe |
| [Lu]DOTA-[α-Me-Trp8]UL | Nle | Phe | His | β-Ala | Val | Ala | α-Me-Trp | Gln | phe |
| [Lu]DOTA-[Bta8]UL | Nle | Phe | His | β-Ala | Val | Ala | Bta | Gln | phe |
| [Lu]DOTA-[Hse7]UL | Nle | Phe | His | β-Ala | Val | Ala | Trp | Hse | phe |
| [Lu]DOTA-[Chg10]MJ9 | Leu | Sta | His | Gly | Chg | Ala | Trp | Gln | phe |
| [Lu]DOTA-[α-Me-Trp8, Chg10]MJ9 | Leu | Sta | His | Gly | Chg | Ala | α-Me-Trp | Gln | phe |
| [Lu]DOTA-[Bta8, Chg10]MJ9 | Leu | Sta | His | Gly | Chg | Ala | Bta | Gln | phe |
| [Lu]DOTA-[Hse7, Chg10]MJ9 | Leu | Sta | His | Gly | Chg | Ala | Trp | Hse | phe |
| [Lu]DOTA-[Cpg10]MJ9 | Leu | Sta | His | Gly | Cpg | Ala | Trp | Gln | phe |
| [Lu]DOTA-[α-Me-Trp8, Cpg10]MJ9 | Leu | Sta | His | Gly | Cpg | Ala | α-Me-Trp | Gln | phe |
| [Lu]DOTA-[Bta8, Cpg10]MJ9 | Leu | Sta | His | Gly | Cpg | Ala | Bta | Gln | phe |
| [Lu]DOTA-[Hse7, Cpg10]MJ9 | Leu | Sta | His | Gly | Cpg | Ala | Trp | Hse | phe |
| [Lu]DOTA-[Ser9]MJ9 | Leu | Sta | His | Gly | Val | Ser | Trp | Gln | phe |
| [Lu]DOTA-[α-Me-Trp8, Ser9]MJ9 | Leu | Sta | His | Gly | Val | Ser | α-Me-Trp | Gln | phe |
| [Lu]DOTA-[Bta8, Ser9]MJ9 | Leu | Sta | His | Gly | Val | Ser | Bta | Gln | phe |
| [Lu]DOTA-[Hse7, Ser9]MJ9 | Leu | Sta | His | Gly | Val | Ser | Trp | Hse | phe |
| [Lu]DOTA-[arg6]MJ9 | Leu | Sta | His | Gly | Val | Ala | Trp | Gln | arg |
| [Lu]DOTA-[arg6, α-Me-Trp8]MJ9 | Leu | Sta | His | Gly | Val | Ala | α-Me-Trp | Gln | arg |
| [Lu]DOTA-[arg6, Bta8]MJ9 | Leu | Sta | His | Gly | Val | Ala | Bta | Gln | arg |
| [Lu]DOTA-[arg6, Hse7]MJ9 | Leu | Sta | His | Gly | Val | Ala | Trp | Hse | arg |
| [Lu]DOTA-[Txa5]MJ9 | Leu | Sta | His | Gly | Val | Ala | Trp | Gln | phe |
| [Lu]DOTA-[Txa5, α-Me-Trp8]MJ9 | Leu | Sta | His | Gly | Val | Ala | α-Me-Trp | Gln | phe |
| [Lu]DOTA-[Txa5, Bta8]MJ9 | Leu | Sta | His | Gly | Val | Ala | Bta | Gln | phe |
| [Lu]DOTA-[Txa5, Hse7]MJ9 | Leu | Sta | His | Gly | Val | Ala | Trp | Hse | phe |
| [Lu]DOTA-orn3-dap4-MJ9 | Leu | Sta | His | Gly | Val | Ala | Trp | Gln | phe |
| [Lu]DOTA-orn3-dap4-[α-Me-Trp8]MJ9 | Leu | Sta | His | Gly | Val | Ala | α-Me-Trp | Gln | phe |
| [Lu]DOTA-orn3-dap4-[Bta8]MJ9 | Leu | Sta | His | Gly | Val | Ala | Bta | Gln | phe |
| [Lu]DOTA-orn3-dap4-[Hse7]MJ9 | Leu | Sta | His | Gly | Val | Ala | Trp | Hse | phe |
| [Lu]NeoBOMB1 | | Di-iPr | His | Gly | Val | Ala | Trp | Gln | phe |
| [Lu]-[α-Me-Trp8]NeoBOMB1 | | Di-iPr | His | Gly | Val | Ala | α-Me-Trp | Gln | phe |
| [Lu]-[Bta8]NeoBOMB1 | | Di-iPr | His | Gly | Val | Ala | Bta | Gln | phe |
| [Lu]-[Hse7]NeoBOMB1 | | Di-iPr | His | Gly | Val | Ala | Trp | Hse | phe |

| Ligands | 5 | 4 | 3 | 2 | 1 |
|---|---|---|---|---|---|
| [Lu]RM2 | Pip | [Lu]DOTA | | | |
| [Lu]AMTG | Pip | [Lu]DOTA | | | |
| [Lu]DOTA-[Bta8]MJ9 | Pip | [Lu]DOTA | | | |
| [Lu]DOTA-[Hse7]MJ9 | Pip | [Lu]DOTA | | | |
| [Lu]DOTA-[N-Me-Gly11]MJ9 | Pip | [Lu]DOTA | | | |
| [Lu]DOTA-[α-Me-Trp8, N-Me-Gly11]MJ9 | Pip | [Lu]DOTA | | | |
| [Lu]DOTA-[Bta8, N-Me-Gly11]MJ9 | Pip | [Lu]DOTA | | | |
| [Lu]DOTA-[Hse7, N-Me-Gly11]MJ9 | Pip | [Lu]DOTA | | | |
| [Lu]UL | [Lu]DOTA | | | | |
| [Lu]DOTA-[α-Me-Trp8]UL | [Lu]DOTA | | | | |
| [Lu]DOTA-[Bta8]UL | [Lu]DOTA | | | | |
| [Lu]DOTA-[Hse7]UL | [Lu]DOTA | | | | |
| [Lu]DOTA-[Chg10]MJ9 | Pip | [Lu]DOTA | | | |
| [Lu]DOTA-[α-Me-Trp8, Chg10]MJ9 | Pip | [Lu]DOTA | | | |
| [Lu]DOTA-[Bta8, Chg10]MJ9 | Pip | [Lu]DOTA | | | |
| [Lu]DOTA-[Hse7, Chg10]MJ9 | Pip | [Lu]DOTA | | | |
| [Lu]DOTA-[Cpg10]MJ9 | Pip | [Lu]DOTA | | | |
| [Lu]DOTA-[α-Me-Trp8, Cpg10]MJ9 | Pip | [Lu]DOTA | | | |
| [Lu]DOTA-[Bta8, Cpg10]MJ9 | Pip | [Lu]DOTA | | | |
| [Lu]DOTA-[Hse7, Cpg10]MJ9 | Pip | [Lu]DOTA | | | |
| [Lu]DOTA-[Ser9]MJ9 | Pip | [Lu]DOTA | | | |
| [Lu]DOTA-[α-Me-Trp8, Ser9]MJ9 | Pip | [Lu]DOTA | | | |
| [Lu]DOTA-[Bta8, Ser9]MJ9 | Pip | [Lu]DOTA | | | |
| [Lu]DOTA-[Hse7, Ser9]MJ9 | Pip | [Lu]DOTA | | | |
| [Lu]DOTA-[arg6]MJ9 | Pip | [Lu]DOTA | | | |
| [Lu]DOTA-[arg6, α-Me-Trp8]MJ9 | Pip | [Lu]DOTA | | | |
| [Lu]DOTA-[arg6, Bta8]MJ9 | Pip | [Lu]DOTA | | | |
| [Lu]DOTA-[arg6, Hse7]MJ9 | Pip | [Lu]DOTA | | | |
| [Lu]DOTA-[Txa5]MJ9 | Txa | [Lu]DOTA | | | |
| [Lu]DOTA-[Txa5, α-Me-Trp8]MJ9 | Txa | [Lu]DOTA | | | |
| [Lu]DOTA-[Txa5, Bta8]MJ9 | Txa | [Lu]DOTA | | | |
| [Lu]DOTA-[Txa5, Hse7]MJ9 | Txa | [Lu]DOTA | | | |
| [Lu]DOTA-orn3-dap4-MJ9 | Pip | dap | orn | [Lu]DOTA | |
| [Lu]DOTA-orn3-dap4-[α-Me-Trp8]MJ9 | Pip | dap | orn | [Lu]DOTA | |
| [Lu]DOTA-orn3-dap4-[Bta8]MJ9 | Pip | dap | orn | [Lu]DOTA | |
| [Lu]DOTA-orn3-dap4-[Hse7]MJ9 | Pip | dap | orn | [Lu]DOTA | |
| [Lu]NeoBOMB1 | PABZA | DIG | [Lu]DOTA | | |
| [Lu]-[α-Me-Trp8]NeoBOMB1 | PABZA | DIG | [Lu]DOTA | | |
| [Lu]-[Bta8]NeoBOMB1 | PABZA | DIG | [Lu]DOTA | | |
| [Lu]-[Hse7]NeoBOMB1 | PABZA | DIG | [Lu]DOTA | | |

Abbreviations used: Bta (3-benzothienyl alanine); Chg (cyclohexyl glycine); Cpg (cyclopentyl glycine); DIG (diglycolic acid); Di-iPr (2,6-dimethylheptan-4-amino); DOTA (1,4,7,10-tetraazacyclododecane-N,N′,N″,N″′-tetraacetic acid); Hse (homoserine); Nle (norleucine); PABZA (p-amino methylaniline); Pip (4-amino-1-carboxymethyl-piperidine); Sta (3S,4S)-4-amino-3-hydroxy-6-methylheptanoic acid); Txa (tranexamic acid).

TABLE 1C

| | | Amount of intact |
|---|---|---|
| | IC₅₀ [nM] ± | tracer after |
| | SD (n = 3), | incubation in |
| | ¹⁷⁷Lu- | human plasma for |
| Ligand | labeled | 72 ± 2 h (n = 2) |
| RM2 | 3.5 ± 0.2 | 33.5 ± 2.7 |
| AMTG | 3.0 ± 0.1 | 77.6 ± 10.1 |
| DOTA-[Bta⁸]MJ9 | 4.6 ± 0.2 | 18.3 ± 2.3 |
| DOTA-[Hse⁷]MJ9 | 19.7 ± 1.6 | 40.1 ± 1.4 |
| DOTA-[N—Me-Gly¹¹]MJ9 | 5.6 ± 0.9 | 6.3 ± 0.6 |
| DOTA-[α-Me-Trp⁸, N—Me-Gly¹¹]MJ9 | 6.4 ± 2.3 | 14.3 ± 1.2 |
| DOTA-[Bta⁸, N—Me-Gly¹¹]MJ9 | 9.2 ± 0.9 | 3.0 ± 0.2 |
| DOTA-[Hse⁷, N—Me-Gly¹¹]MJ9 | 26.4 ± 0.6 | 13.8 ± 0.7 |
| UL | 69.4 ± 3.8 | 4.3 ± 0.2 |
| DOTA-[α-Me-Trp⁸]UL | 101 ± 11 | 10.2 ± 2.7 |
| DOTA-[Bta⁸]UL | 135 ± 11 | 0 ± 0 |
| DOTA-[Hse⁷]UL | 463 ± 211 | 0.9 ± 0.9 |
| DOTA-[Chg¹⁰]MJ9 | 67.3 ± 6.6 | 8.4 ± 0.9 |
| DOTA-[α-Me-Trp⁸, Chg¹⁰]MJ9 | 116 ± 20 | 22.6 ± 1.9 |
| DOTA-[Bta⁸, Chg¹⁰]MJ9 | 95.2 ± 17.4 | 2.5 ± 1.0 |
| DOTA-[Hse⁷, Chg¹⁰]MJ9 | 364 ± 36 | 33.4 ± 1.0 |
| DOTA-[Cpg¹⁰]MJ9 | 36.6 ± 15.9 | 25.3 ± 1.4 |
| DOTA-[α-Me-Trp⁸, Cpg¹⁰]MJ9 | 71.6 ± 6.3 | 25.5 ± 1.9 |
| DOTA-[Bta⁸, Cpg¹⁰]MJ9 | 108 ± 21 | 2.2 ± 0.5 |
| DOTA-[Hse⁷, Cpg¹0]MJ9 | 115 ± 8 | 28.1 ± 2.4 |
| DOTA-[Ser⁹]MJ9 | 19.1 ± 1.9 | 9.8 ± 4.3 |
| DOTA-[α-Me-Trp⁸, Ser⁹]MJ9 | 28.4 ± 3.8 | 22.1 ± 2.2 |
| DOTA-[Bta⁸, Ser⁹]MJ9 | 76.0 ± 2.8 | 1.5 ± 0.2 |
| DOTA-[Hse⁷, Ser⁹]MJ9 | 328 ± 20 | 14.5 ± 0.8 |
| DOTA-[arg⁶]MJ9 | 12.8 ± 0.9 | 13.5 ± 3.4 |
| DOTA-[arg⁶, α-Me-Trp⁸]MJ9 | 48.5 ± 1.6 | 20.1 ± 0.7 |
| DOTA-[arg⁶, Bta⁸]MJ9 | 301 ± 9 | 6.6 ± 2.0 |
| DOTA-[arg⁶, Hse⁷]MJ9 | 405 ± 38 | 18.2 ± 1.9 |
| DOTA-[Txa⁵]MJ9 | 4.4 ± 0.6 | 24.8 ± 0.3 |
| DOTA-[Txa⁵, α-Me-Trp⁸]MJ9 | 11.1 ± 1.3 | 30.6 ± 0.8 |
| DOTA-[Txa⁵, Bta⁸]MJ9 | 8.0 ± 1.4 | 27.1 ± 2.1 |
| DOTA-[Txa⁵, Hse⁷]MJ9 | 205 ± 39 | 26.7 ± 1.3 |
| DOTA-orn³-dap⁴-MJ9 | 1.0 ± 0.0 | 5.9 ± 0.5 |
| DOTA-orn³-dap⁴-[α-Me-Trp⁸]MJ9 | 5.6 ± 1.0 | 16.6 ± 0.8 |
| DOTA-orn³-dap⁴-[Bta⁸]MJ9 | 2.6 ± 0.0 | 2.7 ± 0.3 |
| DOTA-orn³-dap⁴-[Hse⁷]MJ9 | 10.3 ± 2.0 | 7.9 ± 2.5 |
| NeoBOMB1 | 4.2 ± 0.1 | 60.8 ± 1.2 |
| [α-Me-Trp⁸]NeoBOMB1 | 6.8 ± 1.2 | 70.7 ± 2.8 |
| [Bta⁸]NeoBOMB1 | 13.0 ± 3.0 | 36.2 ± 3.8 |
| [Hse⁷]NeoBOMB1 | 8.3 ± 0.7 | 57.4 ± 2.1 |

Preferred compounds of the invention include derivatives of the compounds displayed in Tables 1A and 1B. Said derivatives differ from the compounds of Tables 1A and 1B preferably only in that positions 7 and/or 8 (numbering as in the table) are modified in accordance with this invention.

To give an example, in any of the compounds of Table 1A, tryptophan may be replaced with α-methyl tryptophan, thereby obtaining a preferred compound in accordance with the first and second aspect of this invention.

Similarly, at position 7 of the compounds of Table 1A, Gln (or, where this applies, His or gin) may be replaced with Hse or at position 8 of the compounds of Table 1A, Trp may be substituted by Bta, thereby obtaining a preferred compound in accordance with the third aspect of this invention.

What applies to the particularly preferred modifications of Xaa₁ through Xaa₄ applies mutatis mutandis to any of the modifications at these four positions as disclosed herein above, be it in conjunction with compounds of the first, second or third aspect.

In a fourth aspect—which is also a preferred embodiment of the first and second aspect—the present invention provides a compound of formula (IIIa) or (IIIb)

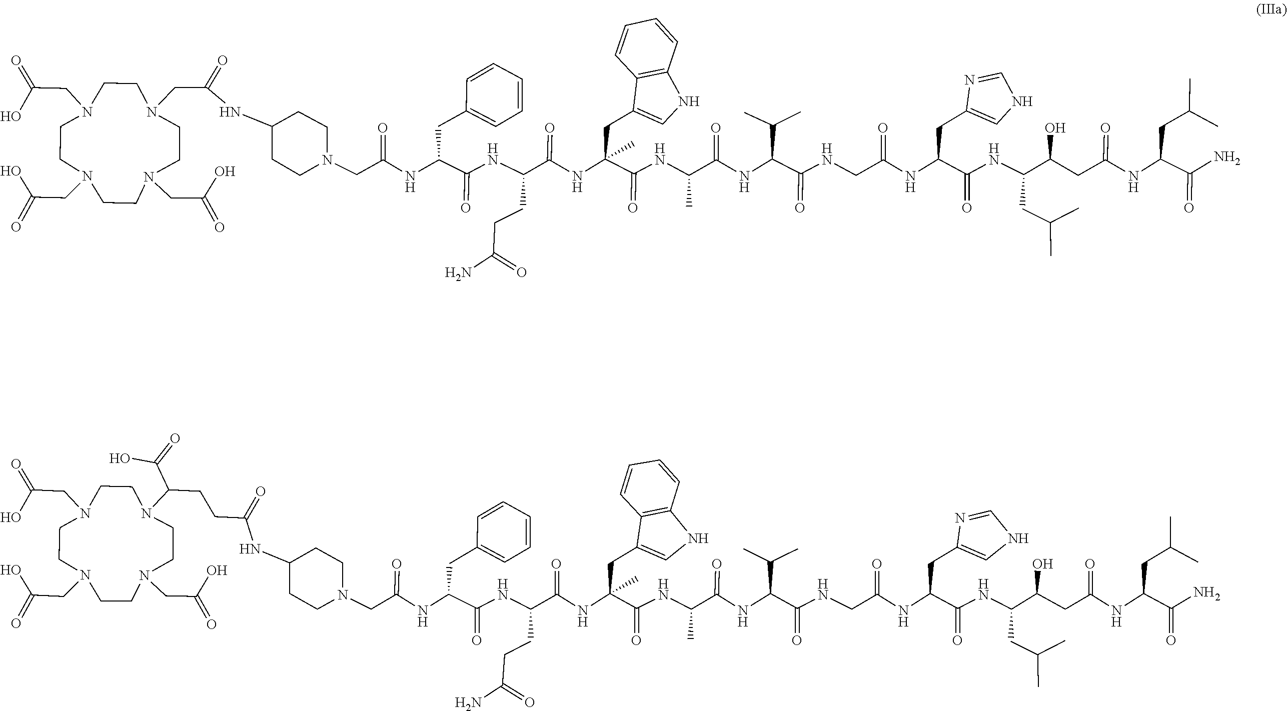

In a fifth aspect—which is also a preferred aspect of the third aspect—the present invention provides a compound of formula (IV) or (V)

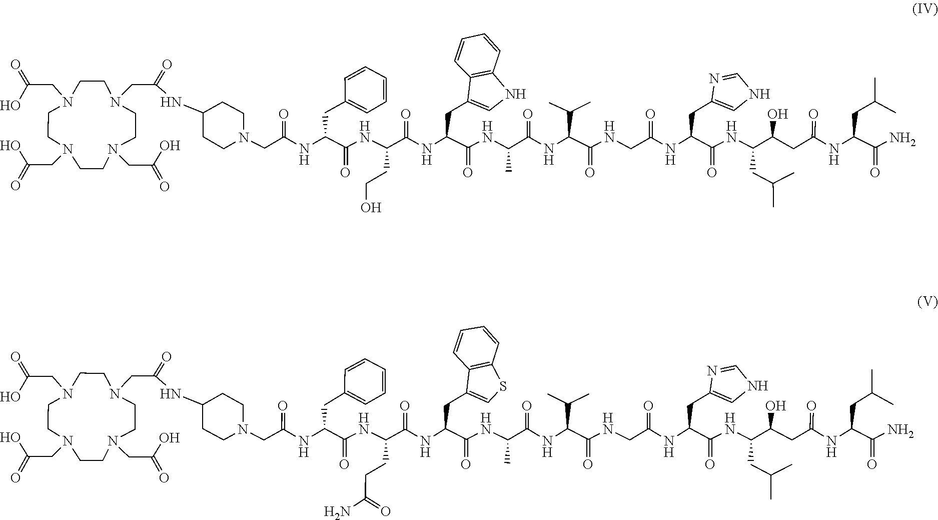 [SMILES: CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCO)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C)C(N)=O ; CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1csc2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C)C(N)=O]

In a sixth aspect, the present invention provides a compound of any of the preceding claims for use in medicine.

In a seventh aspect, the present invention provides a pharmaceutical composition comprising or consisting of a compound of the first, second or fourth aspect.

In an eighth aspect, the present invention provides a diagnostic composition comprising or consisting of a compound of the third or fifth aspect.

Although less preferred, the present invention provides, in a further aspect, a diagnostic composition comprising or consisting of a compound of the first, second or fourth aspect. Also less preferred is a further aspect relating to a pharmaceutical composition comprising or consisting of a compound of the third or fifth aspect.

In the pharmaceutical and diagnostic compositions of the invention, said compound may be the only active agent. It is also possible to make use of more than one compound of the first, second or fourth aspect in the pharmaceutical composition of the invention, and of more than one compound of the third or fifth aspect in the diagnostic composition of the invention.

Also envisaged, although less preferred, are pharmaceutical and diagnostic compositions of the invention wherein in addition to one or more compounds of the present invention further pharmaceutically active or diagnostically active agents are present.

The pharmaceutical or diagnostic composition may further comprise pharmaceutically or diagnostically acceptable carriers, excipients and/or diluents. Examples of suitable carriers, excipients and/or diluents are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical and diagnostic compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration, intravenous being preferred. It is particularly preferred that said administration is carried out by injection. The compositions may also be administered directly to the target site, e.g., by biolistic delivery to an external or internal target site. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

Preferred dosages of radiolabeled (e.g. with ¹⁷⁷Lu) compounds of the invention are from 1 to 100 GBq, 2 to 60 GBq, 2 to 50 GBq, 2 to 10 GBq or 3 to 6 GBq.

Preferred medical indications in accordance with the present invention are hyperproliferative, more preferably malignant diseases.

Accordingly, in a ninth aspect, the present invention provides a pharmaceutical composition of the seventh aspect or a compound of any one of the first, second or fourth aspect, for use in a method of treating cancer, wherein said cancer is (a) characterized by an over-expression of said receptor; and/or (b) selected from prostate cancer, breast cancer, neuroendocrine tumors, Non-Small Cell Lung Cancer (NSCLC), Small-Cell Lung Cancer (SCLC), pancreatic cancer, head/neck squamous cell cancer, neuro/glioblastomas, colorectal cancer and, to the extent said receptor is CCK-2R, Medullary Thyroid Cancer (MTC).

Similarly, the invention provides, in a tenth aspect, a diagnostic composition of the eighth aspect or a compound of the third of fifth aspect for use in a method of diagnosing cancer, wherein said cancer is (a) characterized by an over-expression of said receptor; and/or (b) selected from prostate cancer, breast cancer, neuroendocrine tumors, Non-Small Cell Lung Cancer (NSCLC), Small-Cell Lung Cancer (SCLC), pancreatic cancer, head/neck squamous cell cancer, neuro/glioblastomas, colorectal cancer and, to the extent said receptor is CCK-2R, Medullary Thyroid Cancer (MTC).

In an eleventh aspect, the present invention provides an in vitro method of diagnosing cancer, wherein said cancer is (a) characterized by an over-expression of said receptor; and/or (b) selected from prostate cancer, breast cancer, neuroendocrine tumors, Non-Small Cell Lung Cancer (NSCLC), Small-Cell Lung Cancer (SCLC), pancreatic cancer, head/neck squamous cell cancer, neuro/glioblastomas, colorectal cancer and, to the extent said receptor is CCK-2R, Medullary Thyroid Cancer (MTC), wherein said method comprises bringing into contact a diagnostic composition of the eighth aspect or a compound of the third of fifth aspect, with a sample obtained from the subject.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The present invention includes the following items:

1. A compound binding to an endogenous receptor, said compound comprising
(i) an oligopeptide comprising a dipeptide with Trp being the C-terminal amino acid of said dipeptide, wherein said Trp is replaced with an α-amino acid Xaa₂, whereby the stability in serum or plasma of the peptide bond connecting Xaa₂ to the N-terminally adjacent amino acid is increased as compared to the peptide bond connecting Trp to the N-terminally adjacent amino acid in an otherwise identical compound; and
(ii) a moiety capable of generating therapeutically effective radiation, said moiety being covalently bound to said oligopeptide.

2. The compound of item 1, wherein said N-terminally adjacent amino acid in said dipeptide is L-Gln, D-Gln, L-His, D-His or Gly, preferably L-Gln.

3. The compound of item 1 or 2, wherein said endogenous receptor is a peptide receptor overexpressed in cancer disease, such as Neuromedin-B receptor (Bombesin-1 receptor, NMBR), Gastrin-releasing peptide receptor (Bombesin-2 receptor, GRPR), Bombesin receptor subtype 3 (BRS-3) or Cholecystokinin-2 receptor (CCK-2R), and wherein preferably
(a) said binding is with a Ko of less or equal 15 nM; and/or
(b) said compound is a GRPR antagonist, preferably with an IC₅₀ of less or equal 15 nM.

4. A compound of formula (I)

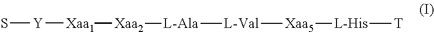

wherein
S is a moiety capable of generating therapeutically active radiation;
Y is an optional linker;
Xaa₁ is (i) L-Gln, D-Gln, L-His, D-His or Gly, preferably L-Gln; or
   (ii) an α-amino acid which increases stability in serum or plasma of the Xaa₁-Xaa₂ peptide bond as compared to Xaa₁ being Gln and Xaa₂ being Trp in an otherwise identical compound;
Xaa₂ is Trp or an α-amino acid which increases stability in serum or plasma of the Xaa₁-Xaa₂ peptide bond as compared to Xaa₁ being Gln and Xaa₂ being Trp in an otherwise identical compound;
provided that Xaa₁ is not any one of L-Gln, D-Gln, L-His, D-His and Gly, and Xaa₂ is not Trp, respectively, at the same time;
Xaa₅ is Gly, N-Me-Gly, D-Ala, β-Ala or 2-aminoisobutyric acid (Aib); preferably Gly; and
T is an optional terminal group.

5. The compound of any one of items 1 to 4, wherein Xaa₂ is
(a) Trp which is modified to comprise
   (i) a C1 to C4 optionally substituted alkyl moiety bound to the α-carbon, substituents being selected from halogen and hydroxyl; and/or
   (ii) a substituent bound to the indole ring, substituents being selected from N-(2,2,2-trifluoromethyl), N-methyl, N-acetyl, 5-fluoro, 5-bromo, 5-iodo, 5-chloro, 5-hydroxy, 5-methoxy, 5-methyl, 6-chloro, 7-chloro and 7-Aza;
(b) 1,2,3,4-tetrahydro norharmane-3-carboxylic acid (L-Tpi).

6. The compound of item 5, wherein said optionally substituted alkyl moiety is selected from —CH₃, —CH₂CH₃, and CHₙHal₃₋ₙ, wherein n is 0, 1 or 2 and Hal is F, Cl, Br and/or I such as —CF₃; and preferably is —CH₃.

7. The compound of any one of items 1 to 6, wherein Xaa₂ is α-Me-Trp.

8. A compound of formula (II)

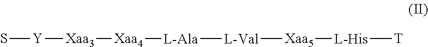

wherein
S is a moiety capable of generating a detectable signal;
Y is an optional linker;
Xaa₃ is (i) L-Gln, D-Gln, L-His, D-His or Gly, preferably L-Gln; or
   (ii) an α-amino acid which decreases stability in serum or plasma of the Xaa₃-Xaa₄ peptide bond as compared Xaa₃ being Gln and Xaa₄ being Trp in an otherwise identical compound;
Xaa₄ is Trp or an α-amino acid which decreases stability in serum or plasma of the Xaa₃-Xaa₄ peptide bond as compared Xaa₃ being Gln and Xaa₄ being Trp in an otherwise identical compound;
wherein said α-amino acid at position Xaa₄ which decreases stability in serum or plasma of the Xaa₃-Xaa₄ peptide bond is not a proteinogenic amino acid;
provided that Xaa₃ is not any one of L-Gln, D-Gln, L-His, D-His and Gly, and Xaa₄ is not Trp, respectively, at the same time;
Xaa₅ is Gly, N-Me-Gly, β-Ala or 2-aminoisobutyric acid (Aib); preferably Gly; and T is a optional terminal group.

9. The compound of item 8, wherein Xaa₃ is Hse and/or Xaa₄ is Bta.

10. The compound of any one of items 4 to 7, wherein S is selected from a radioactive moiety and a moiety capable of being loaded with a radioactive nuclide.

11. The compound of item 8 or 9, wherein S is selected from a fluorescent moiety, a radioactive moiety, and a moiety capable of being loaded with a radioactive nuclide.

12. The compound of item 10 or 11, wherein said moiety capable of being loaded with a radioactive nuclide is a metal ion chelator, preferably selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]hexadecane (DO2A), 1,4,7,10-tetraazacyclododecane-N,N′,N″,N′″-tetraacetic acid (DOTA), α-(2-carboxyethyl)-1,4,7,10-tetraazacyclododecane-1,4,7,10-tetraacetic acid or 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglycol-O,O-bis(2-aminoethyl)-N, N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecane-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridin-bis(methyleneamintetraacetic acid (TMT), 1,4,7,10-tetraazacyclotridecan-N,N′,N″,N′″-tetraacetic acid (TRITA), triethylenetetraaminehexaacetic acid (TTHA), N,N′-bis[(6-carboxy-2-pyridyl)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 6-carboxy-1,4,8,11-tetraazaundecane (N4), 6-{p-[(carboxymethoxy)acetyl]-aminobenzyl}-1,4,8,11-tetraazaundecane (N4′), 1,4,7,10-Tetraazacyclododecane-1,4,7-triacetic acid (DO3A), S-acetylmercaptoacetyltriserine (MAS3), Mercaptoacetyltriglycine (MAG3), 1,4-bis (hydroxycarbonyl methyl)-6-[bis(hydroxylcarbonyl methyl)] amino-6-methyl perhydro-1,4-diazepine (AAZTA), 3,6,9,15-tetraazabicyclo[9.3.1]pentadeca-1 (15), 11,13-triene-2,10-dione (TBPD), 9-oxa-3,6,12,15,21-pentaazatricyclo[15,3,2,1] trieicos-1 (21), 17,19-triene-2,7,11,16-tetradione (OPTT), 2-[Bis(carboxymethyl)aminomethyl]-2-[(4-isothiocyanatobenzyl)oxy-methyl]propylene-1,3-dinitrilotetraacetic Acid (TAME-Hex), 4-((4-(3-(bis(2-(3-hydroxy-1-methyl-2-oxo-1,2-dihydropyridine-4-carboxamido)ethyl)amino)-2-((bis(2-(3-hydroxy-1-methyl-2-oxo-1,2-dihydropyridine-4-carboxamido)ethyl)amino)methyl)propyl)phenyl)amino)-4-oxobutanoic acid (Me-3,2-HOPO), 2,20-(6-((carboxymethyl)amino)-1,4-diazepane-1,4-diyl)diacetic acid) (DATA), 1,4,7-triazacyclononane-1,4,7-tris[methyl (2-carboxyethyl)phosphinic acid] (TRAP) and functional derivatives thereof, such as NOPO (1,4,7-triazacyclononane-1,4-bis[methylene(hydroxymethyl)phosphinic acid]-7-[methylene(2-carboxyethyl)phosphinic acid]), 1,4,7,10-tetraazacyclododecane-1,4,7,10-tetrakis[methylene(2-carboxyethyl)phosphinic acid] (DOTPI), 6,6′-({9-hydroxy-1,5-bis(methoxycarbonyl)-2,4-di(pyridin-2-yl)-3,7-diazabicyclo[3.3.1]nonane-3,7-diyl}bis(methylene))dipicolinic acid (H2bispa2), 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA), 1,2-[{6-(carboxy)-pyridin-2-yl}-methylamino]ethane (H₂dedpa), N,N′-bis{6-carboxy-2-pyridylmethyl}-ethylenediamine-N,N′-diacetic acid (H₄octapa), 4,10-bis(carboxymethyl)-1,4,7,10-tetraazabicyclo[5.5.2]tetradecane (CB-DO2A), 1,4,7,10-tetrakis(carbamoylmethyl)-1,4,7,10-tetraazacyclododecane (TCMC), 1,8-diamino-3,6,10,13,16,19-hexaazabicyclo[6.6.6]icosane (sar) and functional derivatives thereof, {4-[2-(bis-carboxymethylamino)-ethyl]-7-carboxymethyl-[1,4,7]triazonan-1-yl}-acetic acid (NETA), N,N′,N″, tris(2-mercaptoethyl) 1,4,7-triazacyclononane (TACN-TM), 2-(p-isothiocyanatobenzyl)-cyclohexyldiethylenetriaminepentaacetic acid (CHX-A″-DTPA), N,N′-[1-benzyl-1,2,3-triazole-4-yl]methyl-N,N′-[6-(carboxy)pyridin-2-yl]-1,2-diaminoethane (H₂azapa), N,N″-[[6-(carboxy)pyridin-2-yl]methyl]diethylenetriamine-N,N′,N″-triacetic acid (H₅decapa), N,N′-bis(2-hydroxy-5-sulfobenzyl)ethylenediamine-N,N′-diacetic acid (SHBED), 3,6,9,15-tetraazabicyclo[9.3.1]pentadeca-1(15),11,13-triene-3,6,9,-triacetic acid (PCTA), and N,N′-(methylenephosphonate)-N,N′-[6-(methoxycarbonyl)pyridin-2-yl]methyl-1,2-diaminoethane (H₆phospa), more preferably being DOTA or DOTAGA;
wherein preferably a radioactive cation is bound to said chelator,
said radioactive cation preferably being selected from ⁴³Sc, ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹Ag, ^{110m}In, ¹¹¹ In, ^{113m}In, ^{114m}In, ^{117m}Sn, ¹²¹Sn, ¹²⁷Te, ¹⁴⁰La, ¹⁴²La, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁶Dy, ¹⁶⁵Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ¹⁸⁸Re, ¹⁸⁸W, ¹⁹¹ Pt, ^{195m}Pt, ¹⁹⁴Ir, ¹⁹⁷Hg, ¹⁹⁸ Au, ¹⁹⁹ Au, ²¹²Pb, ²⁰³Pb, ²¹¹ At, ²¹²Bi, ²¹³Bi, ²²³Ra, ²²⁴ Ra, ²²⁵Ac, and ²²⁷Th, or a cationic molecule comprising ¹⁸F, such as ¹⁸F-[AIF]²⁺.

13. The compound of any of items 4 to 12, wherein Y is present and
(a) comprises one, two, three, four, five or six positive and/or negative charge(s);
(b) comprises or consists of one, two, three, four, five or six amino acids, preferably (a) D-amino acid(s) being among said amino acids, more preferably (a) D-α-amino acid(s);
(c) comprises or consists of PEGₙ, n being an integer selected from 1, 2, 3, 4, 5, 6, 7, 8, 9 and 10; and/or
(d) comprises a moiety capable of generating a detectable signal.

14. The compound of item 13, wherein said linker Y comprises or consists of
(a) D-Glu-urea-D-Glu;
(b) one or two 2,3-diaminopropionic acid moieties, optionally substituted with a moiety capable of generating a detectable signal;
(c) one, two, three, four, five or six consecutive amino acids comprising or consisting of one or more amino acids selected from D-/L-aspartate, D-/L-ornithine, 4-amino-1-carboxymethyl-piperidine (Pip), D-/L-2,3-diaminopropionic acid, D-/L-serine, D-/L-citrulline moieties, L-cysteic acid (Ala(SO₃H)), amino-valeric acid (Ava), 4-aminobenzoic acid (PABA) and D-Phe; and/or
(d) p-aminomethylaniline-diglycolic acid (pABza-DIG, AMA-DGA), and/or diglycolate (DIG, DGA).

15. The compound of any items 4 to 14, wherein T is present and comprises or consists of
(a) statine (Sta or (3S,4S)-4-amino-3-hydroxy-6-methylheptanoic acid), 2,6-dimethyl heptane, Leu or β-thienyl-L-alanine (Thi);
(b) Leu, norleucine (Nle), Pro, Met, or 1-amino-1-isobutyl-3-methyl-butane, wherein the amidic amine group of said Leu may be modified with ethyl (NH-Ethyl) or NH₂ (NH—NH₂); and/or
(c) (S)-1-((S)-2-amino-4-methylpentyl) pyrrolidine-2-carboxamide (Leu-ψ(CH₂N)-Pro-NH₂);
provided that if T is or terminates with an amino acid, the carboxylate of said amino acid is amidated.

16. The compound of any one of the preceding items, wherein said serum or plasma is human serum or plasma.

17. A compound of formula (IIIa) or (IIIb)

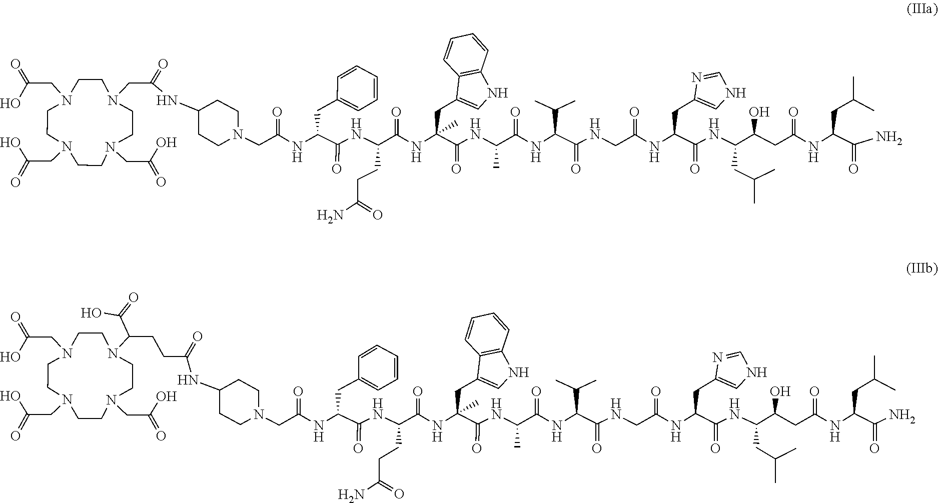

18. A compound of formula (IV) or (V)

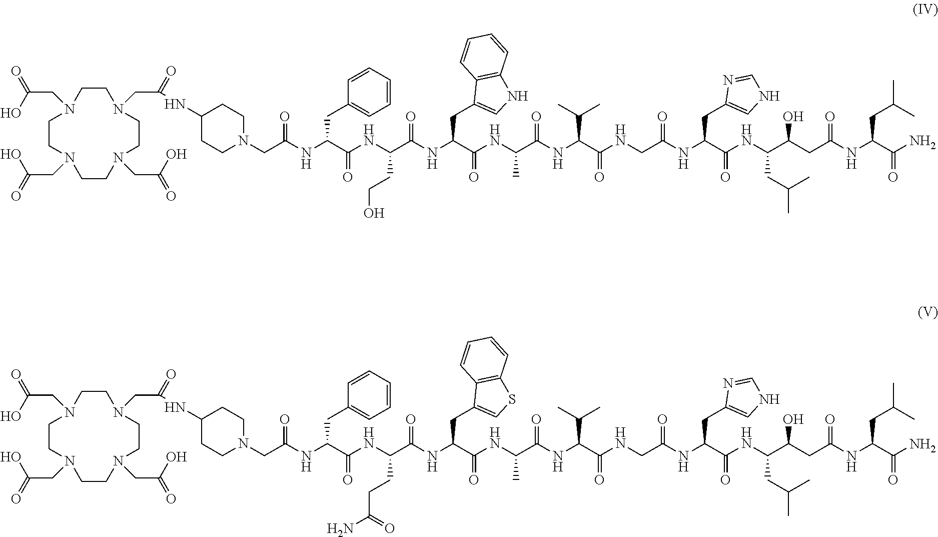 [SMILES: CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCO)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C)C(N)=O ; CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1csc2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C)C(N)=O]

19. A compound of any of the preceding items for use in medicine.

20. A pharmaceutical composition comprising or consisting of a compound of any one of items 1 to 7 or 10 to 17, to the extent items 10 to 16 refer back to any one of items 1 to 7.

21. A diagnostic composition comprising or consisting of a compound of any one of items 8, 9, 10 to 16 or 18, to the extent items 10 to 16 refer back to item 8 or 9.

22. A pharmaceutical composition of item 20 or a compound of any one of items 1 to 7 or 10 to 17, to the extent items 10 to 16 refer back to any one of items 1 to 7, for use in a method of treating cancer, wherein said cancer is
(a) characterized by an over-expression of said receptor; and/or
(b) selected from prostate cancer, breast cancer, neuroendocrine tumors, Non-Small Cell Lung Cancer (NSCLC), Small-Cell Lung Cancer (SCLC), pancreatic cancer, head/neck squamous cell cancer, neuro/glioblastomas, colorectal cancer and, to the extent said receptor is CCK-2R, Medullary Thyroid Cancer (MTC).

23. A diagnostic composition of item 21 or a compound of any one of items 8, 9, 10 to 16 or 18, to the extent items 10 to 16 refer back to item 8 or 9, for use in a method of diagnosing cancer, wherein said cancer is
(a) characterized by an over-expression of said receptor; and/or
(b) selected from prostate cancer, breast cancer, neuroendocrine tumors, Non-Small Cell Lung Cancer (NSCLC), Small-Cell Lung Cancer (SCLC), pancreatic cancer, head/neck squamous cell cancer, neuro/glioblastomas, colorectal cancer and, to the extent said receptor is CCK-2R, Medullary Thyroid Cancer (MTC).

24. An in vitro method of diagnosing cancer, wherein said cancer is
(a) characterized by an over-expression of said receptor; and/or
(b) selected from prostate cancer, breast cancer, neuroendocrine tumors, Non-Small Cell Lung Cancer (NSCLC), Small-Cell Lung Cancer (SCLC), pancreatic cancer, head/neck squamous cell cancer, neuro/glioblastomas, colorectal cancer and, to the extent said receptor is CCK-2R, Medullary Thyroid Cancer (MTC),
wherein said method comprises bringing into contact a diagnostic composition of item 20 or a compound of any one of items 8, 9, 10 to 16 or 18, to the extent items 10 to 16 refer back to item 8 or 9, with a sample obtained from a subject.

The figures show:

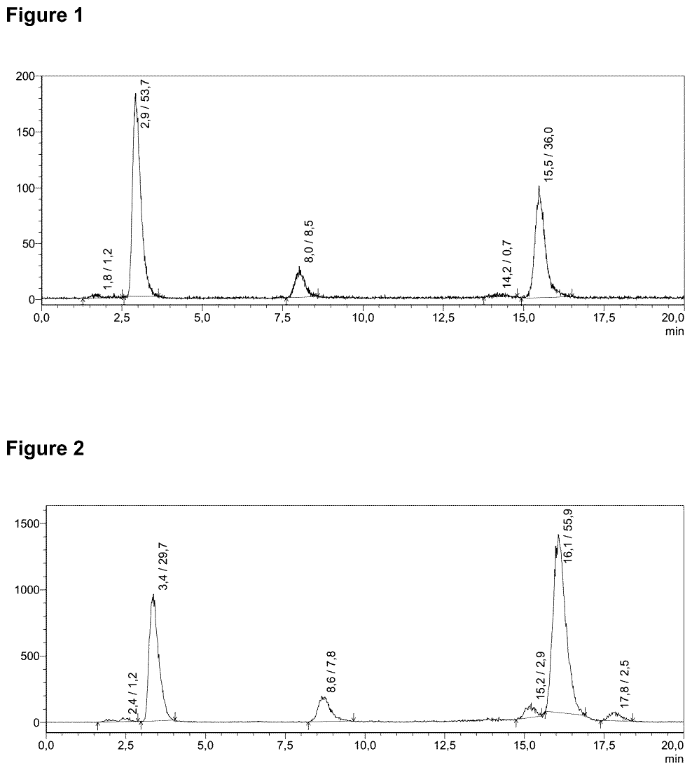

FIG. 1: Analysis of [¹⁷⁷Lu]RM2 (t_{R}=15.3 min, 20→35% in 20 min) after incubation at 37° C. for 72±2 h in human plasma. The chromatogram shows two significant metabolites (t_{R}=2.9 min, 54% and (R=8.5 min, 9%) as well as the remaining intact tracer (t_{R}=15.3 min, 36%).

FIG. 2: Analysis of [¹⁷⁷Lu]DOTA-[Hse⁷]MJ9 (t_{R}=16.1 min, 20→35% in 20 min) after incubation at 37° C. for 72±2 h in human plasma. The chromatogram shows two significant metabolites (t_{R}=3.4 min, 30% and (R=8.6 min, 8%) as well as the remaining intact tracer (t_{R}=15.3 min, 56%).

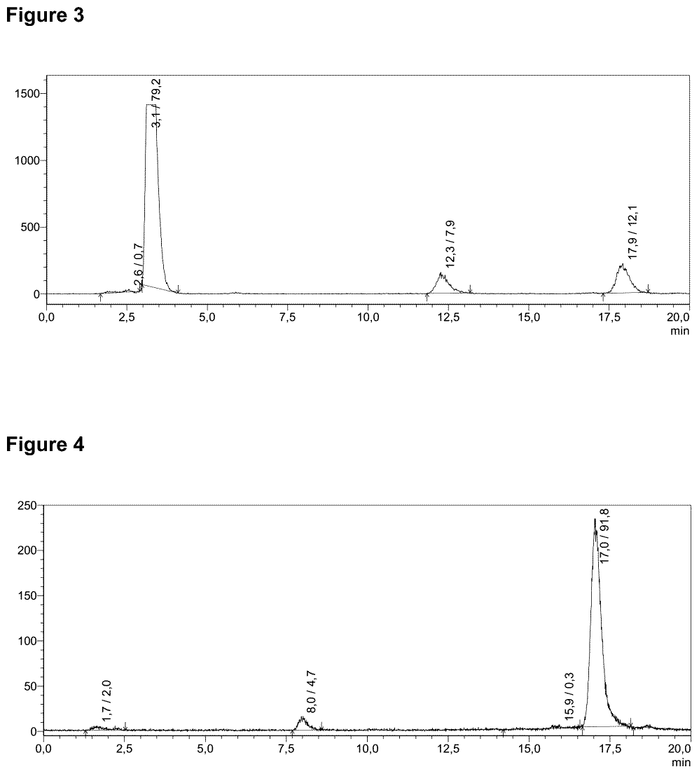

FIG. 3: Analysis of [¹⁷⁷Lu]DOTA-[Bta⁸]MJ9 (t_{R}=17.9 min, 20→35% in 20 min) after incubation at 37° C. for 72±2 h in human plasma. The chromatogram shows two metabolites (t_{R}=3.1 min, 79% and (R=12.3 min, 8%) as well as the remaining intact tracer (t_{R}=17.9 min, 12%).

FIG. 4: Analysis of [¹⁷⁷Lu]AMTG (t_{R}=17.0 min, 20→35% in 20 min) after incubation at 37° C. for 72±2 h in human plasma. The chromatogram shows two metabolites (t_{R}=1.7 min, 2% and (R=8.0 min, 5%) as well as the remaining intact tracer (t_{R}=17.0 min, 92%).

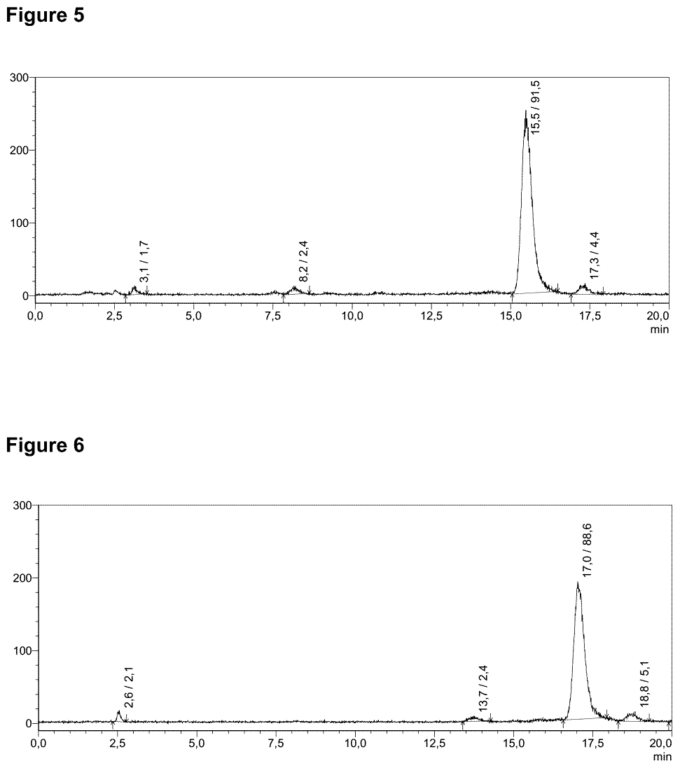

FIG. 5: Analysis of [¹⁷⁷Lu]RM2 (t_{R}=15.5 min, 20→35% in 20 min) after incubation at 37° C. for 6±0.5 h in murine plasma. The chromatogram shows three metabolites (t_{R}=3.1 min, 2%, t_{R}=8.2 min, 2% and t_{R}=17.3 min, 4%) as well as the remaining intact tracer (t_{R}=17.0 min, 92%).

FIG. 6: Analysis of [¹⁷⁷Lu]AMTG ((R=17.0 min, 20→35% in 20 min) after incubation at 37° C. for 6±0.5 h in murine plasma. The chromatogram shows three metabolites (t_{R}=2.6 min, 2%, t_{R}=13.7 min, 2% and t_{R}=18.8 min, 5%) as well as the remaining intact tracer (t_{R}=17.0 min, 89%).

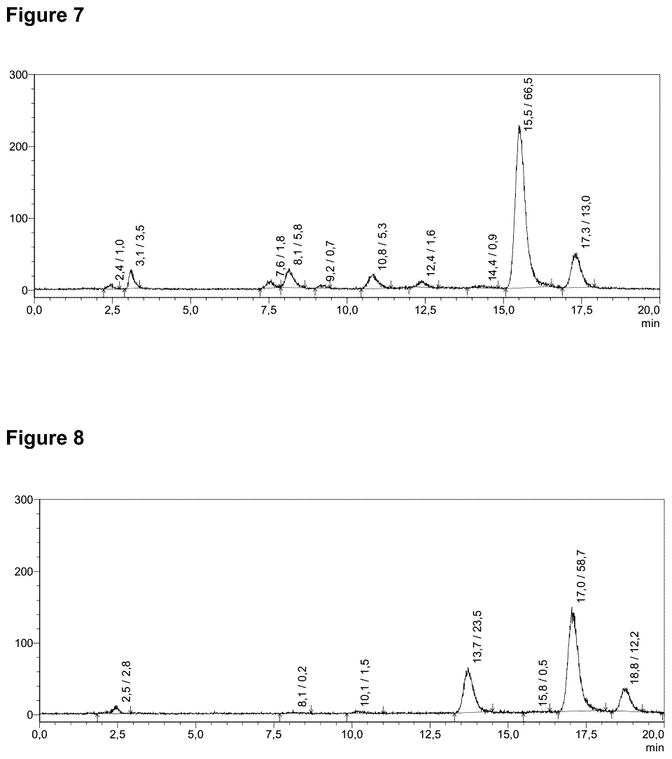

FIG. 7: Analysis of [¹⁷⁷Lu]RM2 (t_{R}=15.5 min, 20→35% in 20 min) after incubation at 37° C. for 72±2 h in murine plasma. The chromatogram shows several small metabolites as well as the remaining intact tracer (t_{R}=15.5 min, 67%).

FIG. 8: Analysis of [¹⁷⁷Lu]AMTG (t_{R}=17.0 min, 20→35% in 20 min) after incubation at 37° C. for 72±2 h in murine plasma. The chromatogram shows several metabolites as well as the remaining intact tracer (t_{R}=17.0 min, 59%).

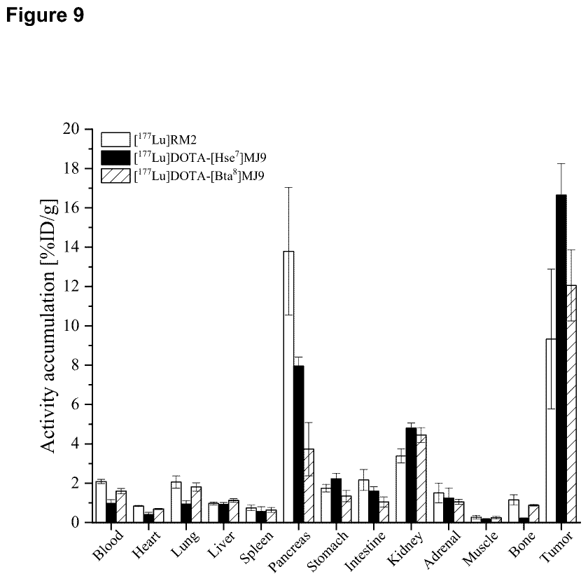

FIG. 9: Biodistribution of [¹⁷⁷Lu]RM2 (white), [¹⁷⁷Lu]DOTA-[Hse⁷]MJ9 (black) and [¹⁷⁷Lu]DOTA-[Bta⁸]MJ9 (hatched) in selected organs (in % ID/g) at 1 h p.i. on PC-3 tumor-bearing CB17-SCID mice (100 pmol each). Data is expressed as mean±SD (n=4).

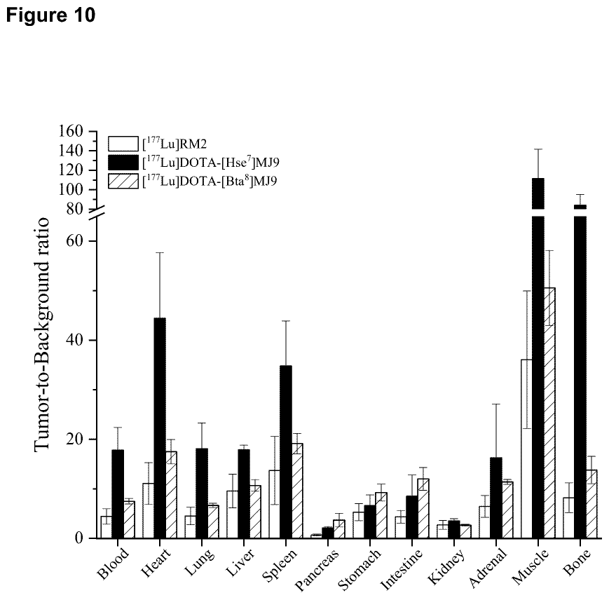

FIG. 10: Tumor-to-background ratios for the selected organs of [¹⁷⁷Lu]RM2 (white), [¹⁷⁷Lu]DOTA-[Hse⁷]MJ9 (black) and [¹⁷⁷Lu]DOTA-[Bta⁸]MJ9 (hatched) at 1 h p.i. on PC-3 tumor-bearing CB17-SCID mice. Data is expressed as mean±SD (n=4).

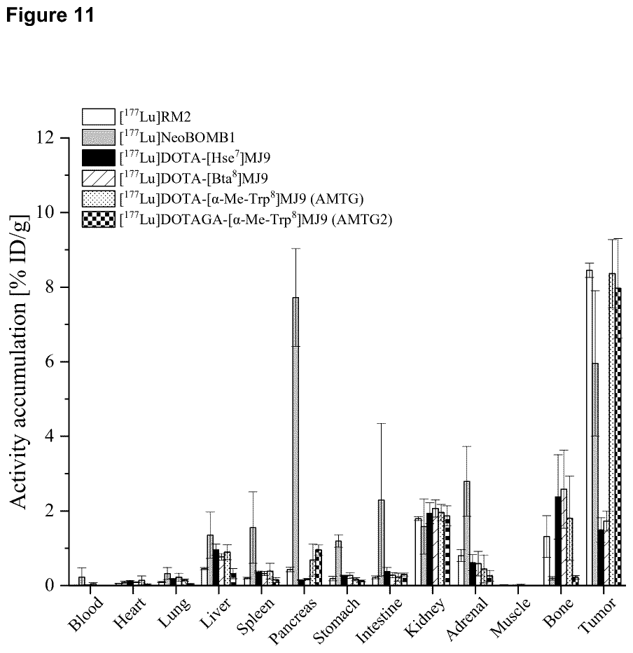

FIG. 11: Biodistribution of [¹⁷⁷Lu]RM2 (white), [¹⁷⁷Lu]NeoBOMB1 (gray), [¹⁷⁷Lu]DOTA-[Hse⁷]MJ9 (black), [¹⁷⁷Lu]DOTA-[Bta⁸]MJ9 (hatched), [¹⁷⁷Lu]AMTG (dotted) and [¹⁷⁷Lu]AMTG2 (squares) in selected organs (in % ID/g) at 24 h p.i. on PC-3 tumor-bearing CB17-SCID mice (100 pmol each). Data is expressed as mean±SD (n=4).

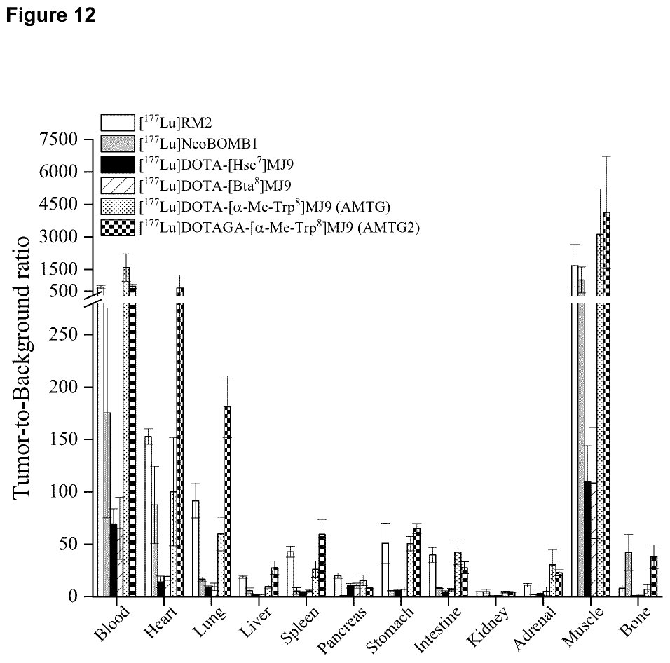

FIG. 12: Tumor-to-background ratios for the selected organs of [¹⁷⁷Lu]RM2 (white), [¹⁷⁷Lu]NeoBOMB1 (gray), [¹⁷⁷Lu]DOTA-[Hse⁷]MJ9 (black), [¹⁷⁷Lu]DOTA-[Bta⁸]MJ9 (hatched), [¹⁷⁷Lu]AMTG (dotted) and [¹⁷⁷Lu]AMTG2 (squares) at 24 h p.i. on PC-3 tumor-bearing CB17-SCID mice. Data is expressed as mean±SD (n=4).

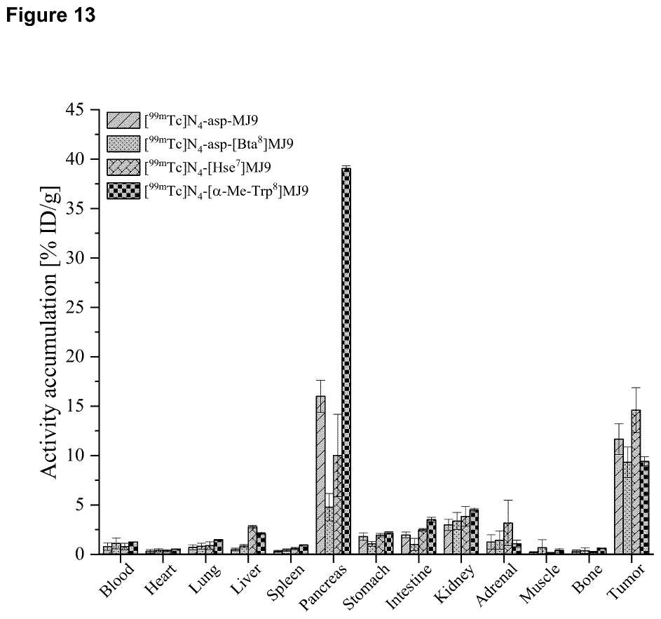

FIG. 13: Biodistribution of [^{99m}Tc]N₄-asp-MJ9 (gray, hatched), [^{99m}Tc]N₄-asp-[Bta⁸]MJ9 (gray, dotted), [^{99m}Tc]N₄-[Hse⁷]MJ9 (gray, bricks) and [^{99m}Tc]N₄-[α-Me-Trp⁸]MJ9 (gray, squares) in selected organs (in % ID/g) at 1 h p.i. on PC-3 tumor-bearing CB17-SCID mice (100 pmol each). Data is expressed as mean±SD (n=4).

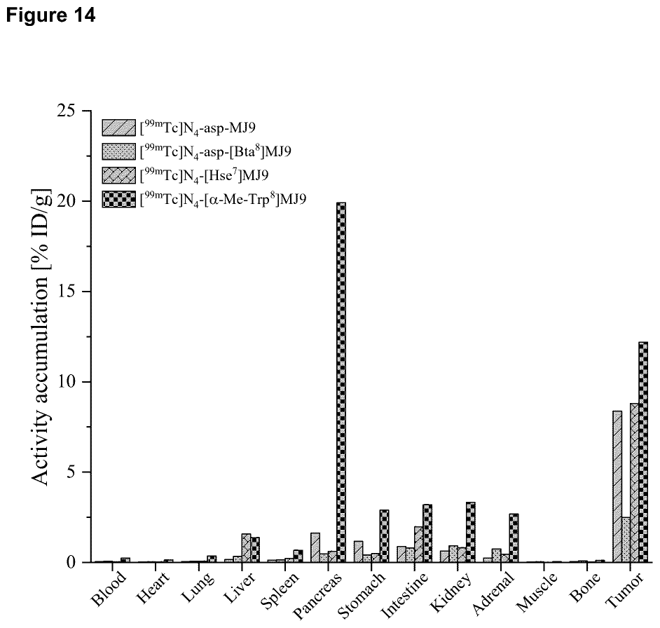

FIG. 14: Biodistribution (n=1) of [^{99m}Tc]N₄-asp-MJ9 (gray, hatched), [^{99m}Tc]N₄-asp-[Bta⁸]MJ9 (gray, dotted), [^{99m}Tc]N₄-[Hse⁷]MJ9 (gray, bricks) and [^{99m}Tc]N₄-[α-Me-Trp⁸]MJ9 (gray, squares) in selected organs (in % ID/g) at 4 h p.i. on PC-3 tumor-bearing CB17-SCID mice (100 pmol each).

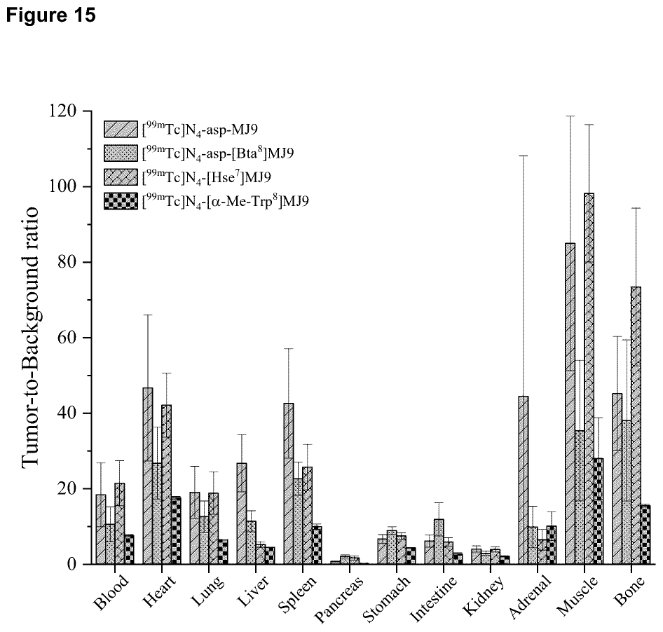

FIG. 15: Tumor-to-background ratios for the selected organs of [^{99m}Tc]N₄-asp-MJ9 (gray, hatched), [^{99m}Tc]N₄-asp-[Bta⁸]MJ9 (gray, dotted), [^{99m}Tc]N₄-[Hse⁷]MJ9 (gray, bricks) and [^{99m}Tc]N₄-[α-Me-Trp⁸]MJ9 (gray, squares) at 1 h p.i. on PC-3 tumor-bearing CB17-SCID mice. Data is expressed as mean±SD (n=4).

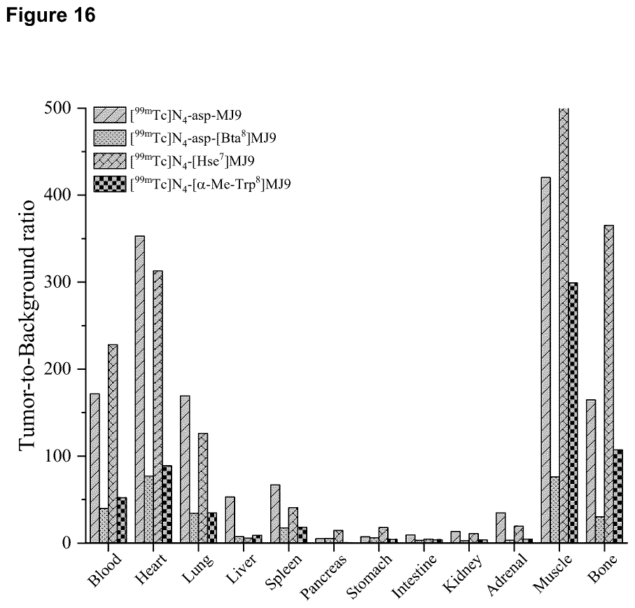

FIG. 16: Tumor-to-background ratios (n=1) for the selected organs of [^{99m}Tc]N₄-asp-MJ9 (gray, hatched), [^{99m}Tc]N₄-asp-[Bta⁸]MJ9 (gray, dotted), [^{99m}Tc]N₄-[Hse⁷]MJ9 (gray, bricks) and [^{99m}Tc]N₄-[α-Me-Trp⁸]MJ9 (gray, squares) at 1 h p.i. on PC-3 tumor-bearing CB17-SCID mice.

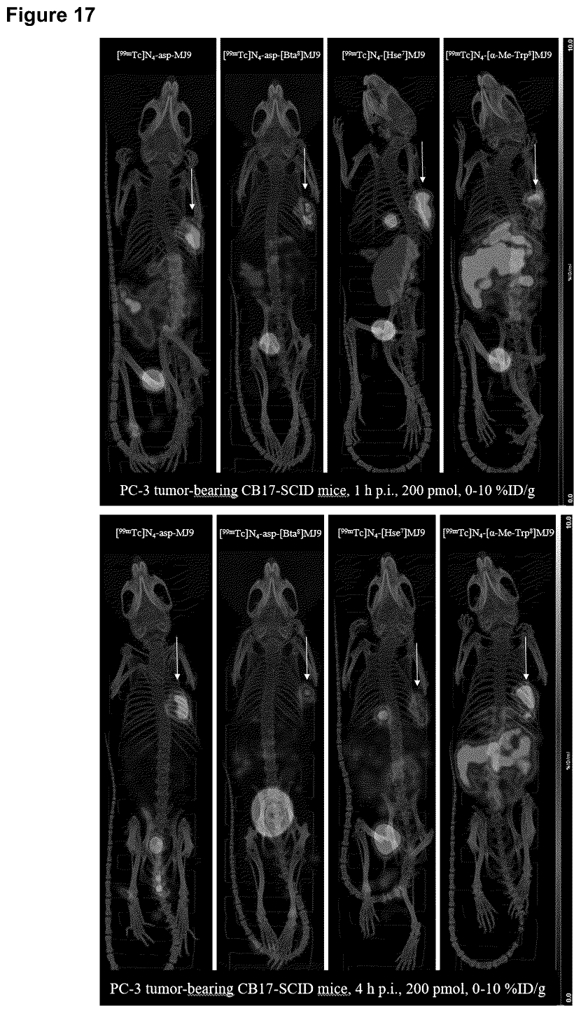

FIG. 17: Maximum intensity projection (dorsal) of [^{99m}Tc]N₄-asp-MJ9, [^{99m}Tc]N₄-asp-[Bta⁸]MJ9, [^{99m}Tc]N₄-[Hse⁷]MJ9 and [^{99m}Tc]N₄-[α-Me-Trp⁸]MJ9 at 1 h p.i. (top) and at 4 h p.i. (bottom) on PC-3 tumor-bearing CB17-SCID mice (200 pmol each). PC-3 tumors are depicted by white arrows.

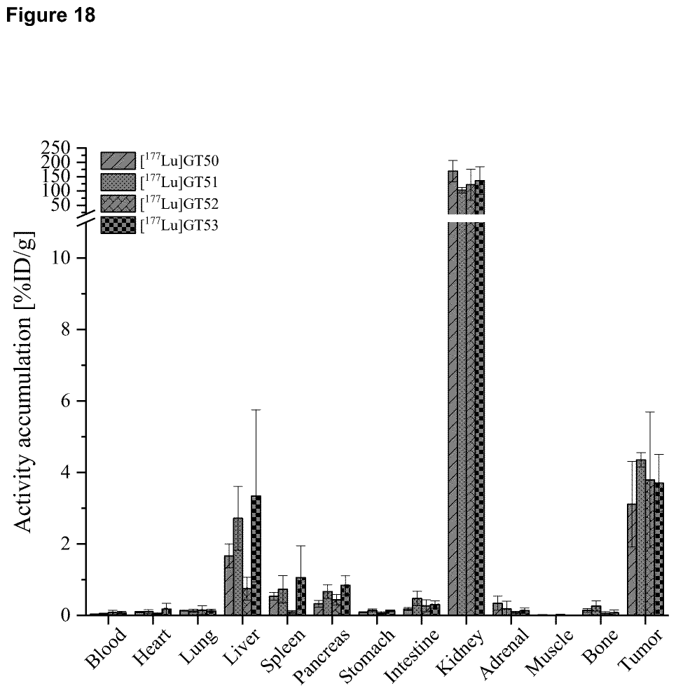

FIG. 18: Biodistribution of [¹⁷⁷Lu]GT50 (dark grey, hatched), [¹⁷⁷Lu]GT51 (dark gray, dotted), [¹⁷⁷Lu]GT52 (dark grey, bricks), [¹⁷⁷Lu]GT53 (dark grey, squares) in selected organs (in % ID/g) at 24 h p.i. on PC-3 tumor-bearing CB17-SCID mice (100 pmol each). Data is expressed as mean±SD (n=4).

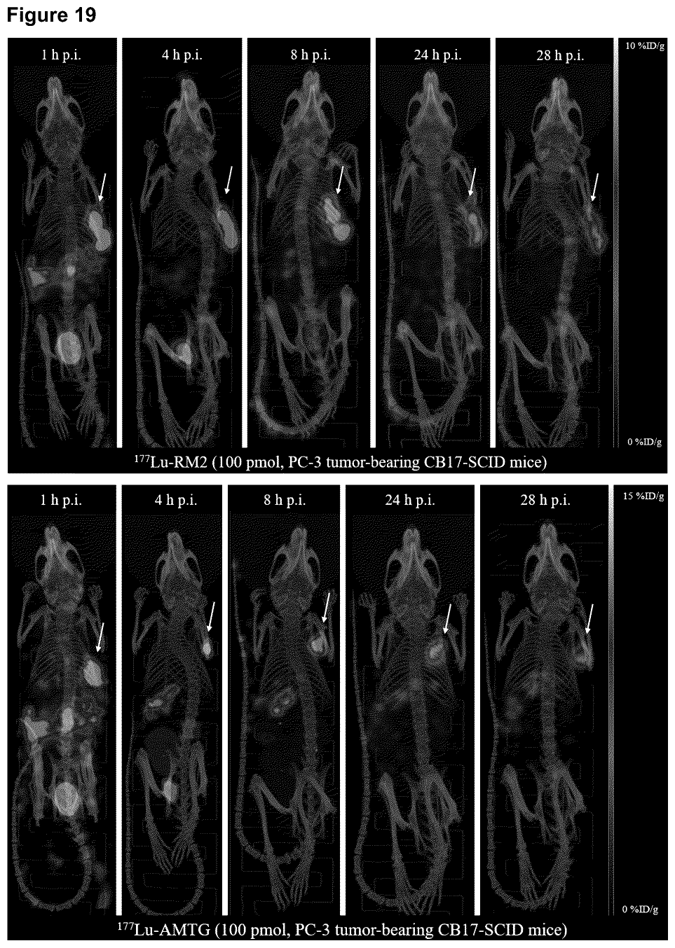

FIG. 19: Maximum intensity projection (dorsal) of [¹⁷⁷Lu]RM2 (top) and [¹⁷⁷Lu]AMTG (bottom) at 1, 4, 8, 24 and 28 h p.i. on PC-3 tumor-bearing CB17-SCID mice (100 pmol each). PC-3 tumors are depicted by white arrows.

The Examples illustrate the invention.

### Example 1: Materials and Methods (General)

The Fmoc-(9-fluorenylmethoxycarbonyl-) and all other protected amino acid analogs are purchased from Bachem (Bubendorf, Switzerland), Sigma-Aldrich (Munich, Germany) or Iris Biotech (Marktredwitz, Germany). The H-Rink amide ChemMatrix® resin (35-100 mesh particle size, 0.4-0.6 mmol/g loading) is purchased from Sigma-Aldrich (Munich, Germany). Chematech (Dijon, France) delivers the chelators DOTA(^{t}Bu)₃ and DOTAGA(^{t}Bu)₄.

All necessary solvents and other organic reagents are purchased from either, Alfa Aesar (Karlsruhe, Germany), Sigma-Aldrich (Munich, Germany) or VWR (Darmstadt, Germany). Solid phase synthesis of the peptides is carried out by manual operation using a Scilogex MX-RL-E Analog Rotisserie Tube Rotator (Scilogex, Rocky Hill, CT, USA).

Analytical and preparative reversed-phase high pressure chromatography (RP-HPLC) are performed using Shimadzu gradient systems (Shimadzu Deutschland GmbH, Neufahrn, Germany), each equipped with a SPD-20A UV/Vis detector (220 nm, 254 nm). Different gradients of acetonitrile (0.1% TFA) in water (0.1% TFA) were used as eluents for all HPLC operations.

For analytical measurements, a Nucleosil 100 C18 (125×4.6 mm, 5 μm particle size) column (CS GmbH, Langerwehe, Germany) is used at a flow rate of 1 mL/min. Both specific gradients and the corresponding retention times t as well as the capacity factor K′ are cited in the text.

Preparative HPLC purification is done with a Multospher 100 RP 18 (250×10 mm, 5 μm particle size) column (CS GmbH, Langerwehe, Germany) at a constant flow rate of 5 mL/min.

Analytical and preparative radio RP-HPLC is performed using a Nucleosil 100 C18 (5 μm, 125×4.0 mm) column (CS GmbH, Langerwehe, Germany).

Electrospray ionization-mass spectra for characterization of the substances are acquired on an expression-CMS mass spectrometer (Advion Ltd., Harlow, UK). Radioactivity is detected through connection of the outlet of the UV-photometer to a NaI(TI) well-type scintillation counter from EG&G Ortec (Munich, Germany).

Radioactive probes are measured by a WIZARD²® 2480 Automatic γ-Counter (Perkin Elmer, Waltham, MA, USA) and determination of IC₅₀ values is carried out using GraphPad Prism 6 (GraphPad Software Inc., San Diego, CA, USA).

For radio TLC, a Scan-RAM™ Scanner with Laura™ software (LabLogic Systems Ltd., Broomhill, Sheffield, United Kingdom) is used.

### Example 2: Synthesis Protocols

### Solid-Phase Peptide Synthesis Following the Fmoc-Strategy

### On-Resin Peptide Formation

The respective side-chain protected Fmoc-AA-OH (1.5 eq.) is dissolved in NMP and pre-activated by adding TBTU (1.5 eq.), HOAt (1.5 eq.) and DIPEA (4.5 eq.). After activation for 10 min, the solution is added to resin-bound free amine peptide and shaken for 1.5 h at room temperature. Subsequently, the resin is washed with NMP and after Fmoc deprotection, the next amino acid is coupled analogously.

### On-Resin Fmoc Deprotection

The resin-bound Fmoc-peptide is treated with 20% piperidine in NMP (v/v) for 5 min and subsequently for 15 min. Afterwards, the resin is washed thoroughly with NMP.

### On-Resin Dde Deprotection

Dde deprotection is performed by adding a solution of imidazole (75 eq.), hydroxylamine hydrochloride (100 eq.) in NMP (7 mL) and DCM (3 mL) for 3 h at room temperature. After deprotection, the resin is washed with NMP.

Conjugation of DOTA(^{t}Bu)₃ or DOTAGA(^{t}Bu)₄

The protected chelator DOTA(^{t}Bu)₃ or DOTAGA(^{t}Bu)₄ (1.5 eq.) is dissolved in NMP and pre-activated by adding TBTU (1.5 eq.), HOAt (1.5 eq.) and DIPEA (4.5 eq.). After activation for 10 min, the solution is added to resin-bound N-terminal deprotected peptide (1.0 eq.) and shaken for 3 h at room temperature. Subsequently, the resin is washed with NMP and DCM.

Peptide Cleavage from the Resin with Additional Deprotection of Acid Labile Protecting Groups

The fully protected resin-bound peptide is washed with DCM, afterwards dissolved in a mixture of TFA/TIPS/DCM (v/v/v; 95/2.5/2.5) and shaken for 30 min. The solution is filtered off and the resin is treated in the same way for another 30 min. Both filtrates are combined and concentrated under a stream of nitrogen. After dissolving the residue in MeOH and precipitation in diethyl ether, the liquid is decanted and the remaining solid is dried.

Deprotection of Remaining ^{t}Bu/Boc

Removal of remaining Bu/Boc protecting groups after peptide cleavage from the resin (see above) is carried out by dissolving the crude product in TFA and stirring for 6 h at room temperature. After removing TFA under a stream of nitrogen, the crude unprotected product is obtained.

### Example 3: Materials and Methods (Labeling Experiments)

### Cold Complexations

[^{nat}Ga]Gallium Complexation

The purified chelator-containing ligand (10⁻³ M in Tracepur H₂O, 1.00 eq.) and [^{nat}Ga]Ga(NO₃)₃·6 H₂O (10 mm in Tracepur H₂O, 1.50 eq.) are diluted with Tracepur water to a final concentration of 10⁻⁴ M and heated to 70° C. for 30 min. After cooling to room temperature, the crude product is obtained.

[^{nat}Lu]Lutetium Complexation

The purified chelator-containing ligand (10⁻³ M in Tracepur H₂O, 1.00 eq.) and [^{nat}Lu]LuCl₃ (20 mm in Tracepur H₂O, 2.50 eq.) are diluted with Tracepur water to a final concentration of 10⁻⁴ M and heated to 95° C. for 30 min. After cooling to room temperature, the crude product is obtained.

### Radiolabeling

### [¹²⁵I]Iodine Labeling

The reference ligand for IC₅₀ studies ([D-3-[¹²⁵I]I-Tyr⁶]MJ9) is prepared according to a previously published procedure. Briefly, 0.2 mg of [D-Tyr⁶]MJ9 are dissolved in 20 μL Tracepur water and 280 μL TRIS buffer (25 mm TRIS HCl, 0.4 M NaCl, pH=7.9). After addition of the solution to a vial containing 150 μg Iodo-Gen® (1,3,4,6-Tetrachloro-3α,6α-diphenylglycouril, surface-bound), 5.0 μL (16 MBq) [¹²⁵I]Nal(74 TBq/mmol, 3.1 GBq/mL, 40 mM NaOH, Hartmann Analytic, Braunschweig, Germany) are added. The reaction solution was incubated for 15 min at room temperature and purified by RP-HPLC (20→35% in 20 min): t_{R}=18.9 min, K′=10.46.

### [¹⁷⁷Lu]Lutetium Labeling

Labeling with [¹⁷⁷Lu]Lutetium is done by using a procedure developed within the group. Therefore, a solution of the purified chelator-containing ligand (10⁻³ M in Tracepur H₂O, 1 μL), sodium acetate buffer (1 M, PH=5.50, 10 μL) and approximately 10⁻³⁰ MBq [¹⁷⁷Lu]LuCl₃ (0.04 M in HCl) are diluted with HCl (0.04 M) to a total volume of 90 μL and heated to 95° C. for 10 min. Immediately after labeling, sodium ascorbate (0.1 M, 10 μL) is added to prevent radiolysis. Incorporation of the [¹⁷⁷Lu]Lutetium is determined by radio TLC (ITLC-SG chromatography paper, mobile phase: 0.1 M trisodium citrate). Radiochemical purity of the labeled compound is determined by radio RP-HPLC.

[^{99m}Tc]Technetium Labeling

Labeling with [^{99m}Tc]technetium is accomplished using a procedure developed within the group. Therefore, a solution of the purified chelator-containing ligand (10⁻³ M in Tracepur H₂O, 5 μL), NaHPO₄ buffer (0.05 M, pH=11.5, 25 μL), sodium citrate buffer (0.1 M, 3 μL), SnCl₂ solution (1 g/L in sodium ascorbate solution (3 g/L), 5 μL) and approximately 50-150 MBq [^{99m}TcO₄]⁻ are heated to 95° C. for 10 min. Incorporation of the [^{99m}Tc]technetium is determined by radio TLC (ITLC-SG chromatography paper, mobile phase: isotonic NaCl). Radiochemical purity of the labeled compound is determined by radio RP-HPLC.

### Example 4: Materials and Methods (In Vitro Experiments)

n-Octanol-PBS Distribution Coefficient, Log D_{7.4}

Approximately 1 MBq of the labeled tracer was dissolved in 1 mL of a 1:1 mixture (v/v) of phosphate buffered saline (PBS, pH=7.4) and n-octanol in an Eppendorf tube. After vigorous mixing of the suspension for 3 min at room temperature, the vial was centrifuged at 9000 rpm for 5 min (Biofuge 15, Heraus Sepatech, Osterode, Germany) and 200 μL aliquots of both layers were measured in a γ-counter. The experiment was repeated at least four times.

### Determination of IC₅₀

The GRPR-positive PC-3 cells are cultured in Dublecco modified Eagle medium/Nutrition Mixture F-12 with Glutamax-I (1:1) (Invitrigon), supplemented with 10% fetal calf serum and maintained at 37° C. in a humidified 5% CO₂ atmosphere. For determination of the GRPR affinity (IC₅₀), cells are harvested 24±2 h before the experiment and seeded in 24-well plates (1.5×10⁵ cells in 1 mL/well).

After removal of the culture medium, the cells are washed once with 500 μL of HBSS (Hank's balanced salt solution, Biochrom, Berlin, Germany, with addition of 1% bovine serum albumin (BSA)) and left in 200 μL HBSS (1% BSA) for 9 min at room temperature for equilibration. Next, 25 μL per well of solutions, containing either HBSS (1% BSA) as control or the respective ligand in increasing concentration (10⁻¹⁰ M-10⁻⁴ M in HBSS), are added with subsequent addition of 25 μL of [D-3-[¹²⁵I]I-Tyr⁶]MJ9 (2.0 nM) in HBSS (1% BSA).

All experiments are performed in triplicate for each concentration. After 2 h incubation at room temperature, the experiment is terminated by removal of the medium and consecutive rinsing with 300 μL of HBSS. The media of both steps are combined in one fraction and represent the amount of free radiolabeled reference. Afterwards, the cells are lysed with 300 μL of 1 M NaOH for at least 15 min and united with the 300 μL NaOH of the following washing step. Quantification of bound and free radiolabeled reference is accomplished in a γ-counter.

IC₅₀ determination for each ligand was repeated twice.

### Internalization

For internalization studies, PC-3 cells are harvested 24±2 h before the experiment and seeded in 24-well plates (1.5×10⁵ cells/well, 1 mL). Subsequent to the removal of the culture medium, the cells are washed once with 500 μL DMEM/F-12 (5% BSA) and left to equilibrate at 37° C. for at least 15 min in 200 μL DMEM/F-12 (5% BSA). Each well is treated with either 25 μL of DMEM/F-12 (5% BSA) or 25 μL [^{nat}Lu]RM2 (10⁻³ M) for blockade. Next, 25 μL of the ¹²⁵I/¹⁷⁷Lu-labeled GRPR ligand (10 nm) is added and the cells are incubated at 37° C. for 60 min.

The experiment is terminated by placing the 24-well plate on ice for 1 min and consecutive removal of the medium. Each well is rinsed with 300 μL ice-cold PBS and the fractions from these first two steps are combined, representing the amount of free radiolabeled reference. Removal of surface bound activity is accomplished by incubation of the cells with 300 μL of ice-cold Acid Wash solution (0.02 M NaOAc, pH=5.0) for 10 min at room temperature and rinsed again with 300 μL of ice-cold PBS. The internalized activity is determined by incubation of the cells in 300 L NaOH (1 M) and the combination with the fraction of a subsequent washing step with 300 μL NaOH (1 M).

Each experiment (control and blockade) is performed sixfold. Free, surface bound and internalized activity is quantified in a γ-counter. Data is corrected for non-specific internalization.

### Plasma Studies

Metabolic stability in vitro was determined applying a procedure published by Linder et al. that was slightly modified. Immediately after labeling, human (200 μL) or murine (100 μL) plasma was added and the mixture was incubated at 37° C. for 72±2 h (or 6±0.5 h). Proteins were precipitated by treatment with ice-cold EtOH (150 μL [human], 100 μL [murine]) and ice-cold MeCN (450 μL [human], 300 μL [murine]), followed by centrifugation for 20 min at 13000 rpm. The supernatants were decanted and further analyzed using radio RP-HPLC.

### Example 5: Materials and Methods (In Vivo Experiments)

All animal experiments were conducted in accordance with general animal welfare regulations in Germany (German animal protection act, as amended on 18 May 2018, Art. 141 G v. 29.3.2017 | 626, approval no. ROB-55.2-2532.Vet_02-18-109) and the institutional guidelines for the care and use of animals. To establish tumor xenografts, PC-3 cells (5×10⁶ cells per 200 μL) were suspended in a 1:1 mixture (v/v) of Dulbecco's modified eagle's medium/Ham's F-12 (DMEM/F-12) with Glutamax-I (1:1) and Cultrex® Basement Membrane Matrix Type 3 (Trevigen Inc., Gaithersburg, MD, USA) and inoculated subcutaneously onto the right shoulder of 6-10 weeks old female CB17-SCID mice (Charles River Laboratories International Inc., Sulzfeld, Germany). Mice were used for experiments when tumor volume was 125-500 mm³ (2-3 weeks after inoculation).

### Biodistribution

Approximately 1-5 MBq (100-200 μmol) of the radiolabeled GRPR antagonists were injected into the tail vein of PC-3 tumor-bearing mice and sacrificed at 1, 4 or 24 h p.i. (n=4). Selected organs were removed, weighted and measured in a γ-counter (Perkin Elmer, Waltham, MA, USA).

### μSPECT/CT Imaging

Imaging studies were performed at a MILabs VECTor⁴ small-animal SPECT/PET/OI/CT device (MILabs, Utrecht, the Netherlands). Data were reconstructed using the MILabs Rec software (version 10.02) and a pixel-based Similarity-Regulated Ordered Subsets Expectation Maximization (SROSEM) algorithm, followed by data analysis using the PMOD4.0 software (PMOD TECHNOLOGIES LLC, Zurich, Switzerland). For SPECT studies mice were anesthetized with isoflurane and injected with 2-4 MBq (100-200 μmol) of the radiolabeled tracer into the tail vein. Static images were recorded within 1 and 28 h p.i. with an acquisition time of 45-60 min using the HE-GP-RM collimator and a step-wise multi-planar bed movement.

### Example 6: Results

### GRPR Reference Ligands

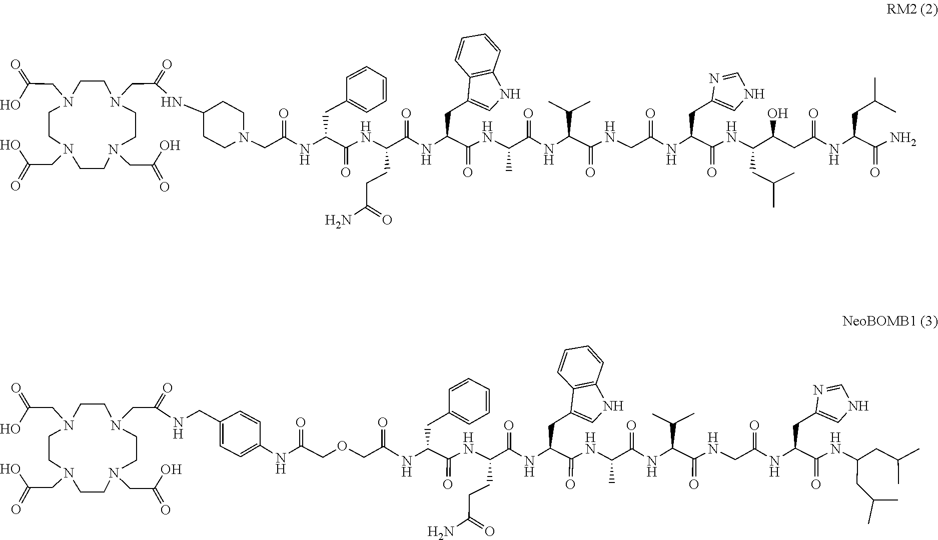 [SMILES: CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C)C(N)=O ; CC(C)CC(CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

### Exemplary Synthesized Antagonistic GRPR Ligands of the Invention

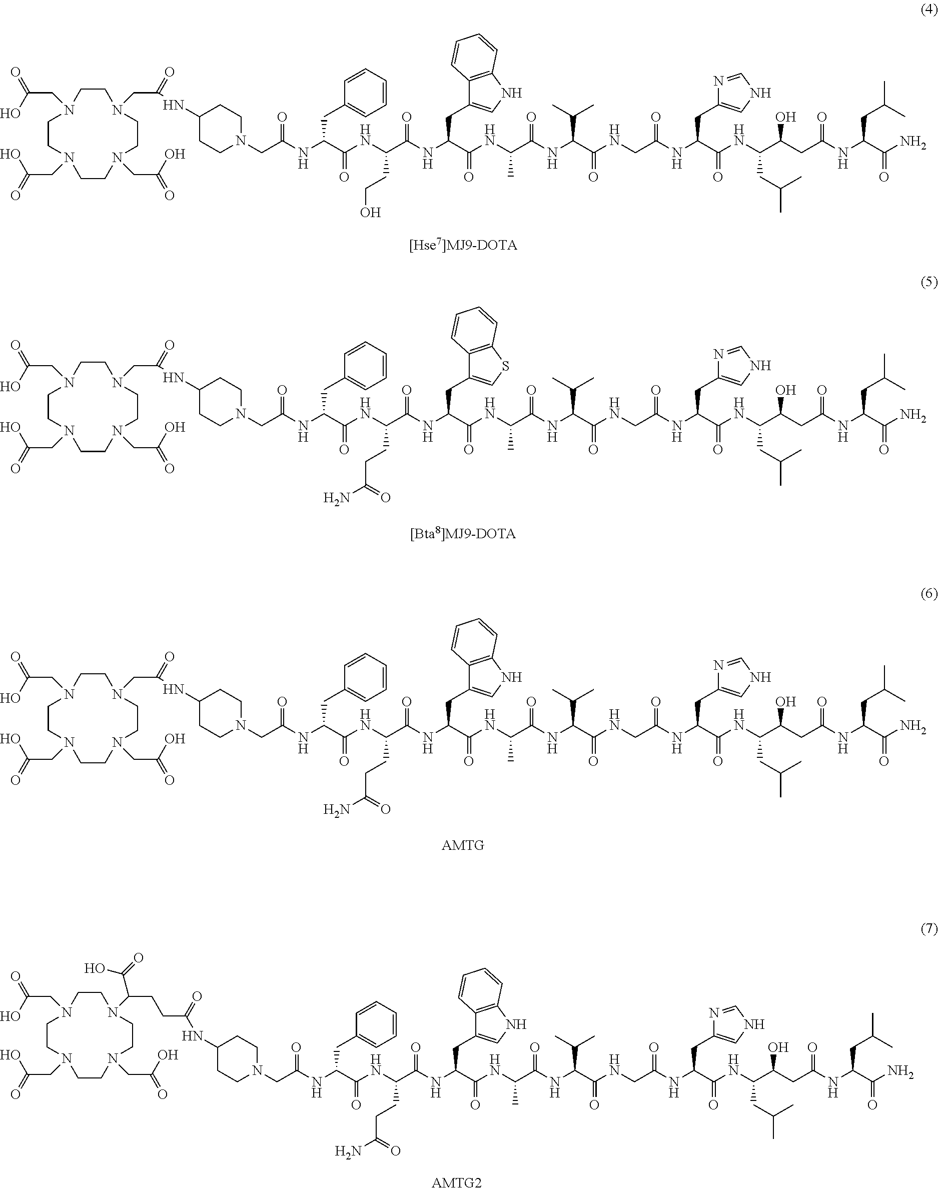

### HPLC

[^{nat}Ga]RM2 (10→90% MeCN in 15 min): t_{R}=6.7 min, K′=3.47.

Calculated monoisotopic mass (C₇₈H₁₁₅GaN₂₀O₁₉): 1704.8, found: m/z=1706.6 [M+H]⁺, 854.1 [M+2H]²⁺.

[^{nat}Ga]DOTA-[Hse⁷]MJ9 (10→90% MeCN in 15 min): t_{R}=6.8 min, K′=3.53.

Calculated monoisotopic mass (C₇₇H₁₁₄GaN₁₉O₁₉): 1677.8, found: m/z=1679.3 [M+H]⁺, 840.4 [M+2H]²⁺.

[^{nat}Ga]DOTA-[Bta⁸]MJ9 (10→90% MeCN in 15 min): t_{R}=7.0 min, K′=3.67.

Calculated monoisotopic mass (C₇₈H₁₁₄GaN₁₉O₁₉S): 1721.7, found: m/z=1723.7 [M+H]⁺, 862.3 [M+2H]²⁺.

[^{nat}Ga]AMTG (10→90% MeCN in 15 min): t_{R}=6.9 min, K′=3.60.

Calculated monoisotopic mass (C₇₉H₁₁₇GaN₂₀O₁₉): 1718.8, found: m/z=1720.0 [M+H]⁺, 860.6 [M+2H]²⁺.

[^{nat}Ga]AMTG2 (10→90% MeCN in 15 min): t_{R}=6.9 min, K′=3.31.

Calculated monoisotopic mass (C₈₂H₁₂₁GaN₂₀O₂₁): 1790.8, found: m/z=896.3 [M+2H]²⁺, 1792.6 [M+H]⁺.

[^{nat=}Lu]RM2 (10→90% MeCN in 15 min): t_{R}=6.6 min, K′=3.40.

Calculated monoisotopic mass (C₇₈H₁₁₅LuN₂₀O₁₉): 1810.8, found: m/z=1812.2 [M+H]⁺, 906.8 [M+2H]²⁺.

[^{nat}Lu]DOTA-[Hse⁷]MJ9 (10→90% MeCN in 15 min): t_{R}=6.8 min, K′=3.53.

Calculated monoisotopic mass (C₇₇H₁₁₄LuN₁₉O₁₉): 1783.8, found: m/z=1784.9 [M+H]⁺, 893.6 [M+2H]²⁺.

[^{nat}Lu]DOTA-[Bta⁸]MJ9 (10→90% MeCN in 15 min): t_{R}=7.0 min, K′=3.67.

Calculated monoisotopic mass (C₇₈H₁₁₄LuN₁₉O₁₉S): 1827.8, found: m/z=1828.9 [M+H]⁺, 915.1 [M+2H]²⁺.

[^{nat}Lu]AMTG (10→90% MeCN in 15 min): t_{R}=6.8 min, K′=3.53.

Calculated monoisotopic mass (C₇₉H₁₁₇LuN₂₀O₁₉): 1824.8, found: m/z=1826.3 [M+H]⁺, 913.6 [M+2H]²⁺.

[^{nat}Lu]AMTG2 (10→90% MeCN in 15 min): t_{R}=7.0 min, K′=3.38.

Calculated monoisotopic mass (C₈₂H₁₂₁LuN₂₀O₂₁): 1896.8, found: m/z=949.5 [M+2H]²⁺, 1897.6 [M+H]⁺.

[^{nat}Lu]NeoBOMB1 (10→90% MeCN in 15 min): t_{R}=9.6 min, K′=5.00. Calculated monoisotopic mass (C₇₇H₁₀₇LuN₁₈O₁₈): 1746.7, found: m/z=874.5 [M+2H]²⁺, 1747.3 [M+H]⁺.

Determination of Hydrophilicity (n-Octanol-PBS Distribution Coefficient, Log D_{7.4})

The determined n-octanol/PBS distribution coefficients (log D_{7.4}) of the ¹⁷⁷Lu-labeled compounds are presented in Table 2. For all compounds, either DOTA or DOTAGA was used as a chelator. Within the ¹⁷⁷Lu-labeled GRPR ligands, the reference RM2 was found to be the most hydrophilic whereas the 3-benzothienyl alanine (Bta) modified derivative was the most lipophilic.

TABLE 2
Distribution coefficients (log D_{7.4} values) of the radiolabeled GRPR
ligands. Data are expressed as mean ± SD (n = 8).

| ¹⁷⁷Lu-labeled ligand | log D_{7.4} ± SD (n = 8) |
|---|---|
| RM2 | −2.51 ± 0.02 |
| DOTA-[Hse⁷]MJ9 | −2.25 ± 0.06 |
| DOTA-[Bta⁸]MJ9 | −1.81 ± 0.02 |
| AMTG | −2.28 ± 0.06 |
| AMTG2 | −2.51 ± 0.11 |
| NeoBOMB1 | −0.57 ± 0.03 |

### Determination of GRPR Affinities

The synthesized compounds showed affinities in a comparable range while the homoserine derivatives had slightly decreased affinities. All [^{nat}Ga]Gallium complexed ligands yielded higher affinities as their [^{nat}Lu]Lutetium complexed counterparts (Table 3). The cold standard [D-3-I-Tyr⁶]MJ9 showed particularly high affinity suggesting its suitability as competing radiolabeled reference for all IC₅₀ experiments.

TABLE 3
Binding affinities of the synthesized Bombesin antagonists
to GRPR. Affinities were determined using PC-3 cells (1.5 ×
10⁵ cells/well) and [D-3-[¹²⁵I]I-Tyr⁶]MJ9 (c =
0.2 nM) as the radiolabeled reference (2 h, r.t., HBSS +
1% BSA). Data are expressed as mean ± SD (n = 3).

| GRPR ligand | IC₅₀ ± SD (n = 3) |
|---|---|
| [D-3-I-Tyr⁶]MJ9 | 1.33 ± 0.36 |
| [^{nat}Ga]RM2 | 1.94 ± 0.16 |
| [^{nat}Ga]DOTA-[Hse⁷]MJ9 | 12.2 ± 0.2 |
| [^{nat}Ga]DOTA-[Bta⁸]MJ9 | 3.06 ± 0.15 |
| [^{nat}Ga]AMTG | 3.28 ± 0.33 |
| [^{nat}Ga]AMTG2 | 5.82 ± 0.23 |
| [^{nat}Lu]RM2 | 3.45 ± 0.18 |
| [^{nat}Lu]DOTA-[Hse⁷]MJ9 | 19.7 ± 1.6 |
| [^{nat}Lu]DOTA-[Bta⁸]MJ9 | 4.63 ± 0.23 |
| [^{nat}Lu]AMTG | 3.04 ± 0.08 |
| [^{nat}Lu]AMTG2 | 4.74 ± 0.23 |
| [^{nat}Lu]NeoBOMB1 | 4.22 ± 0.14 |

### Internalization

In order to proof the antagonistic character of the modified statine-based GRPR ligands, internalization into PC-3 cells was determined. All ¹⁷⁷Lu-labeled compounds showed low internalization as expected from antagonists (Table 4). Internalization of [¹⁷⁷Lu]RM2 showed good correlation with results of other published studies.

TABLE 4
Summary of the internalized activity (c = 1 nM) at 1 h
as % of the used activity, determined on PC-3 cells (37° C.,
DMEM/F-12 + 5% BSA, 1.5 × 10⁵ cells/well). Data is corrected for
non-specific binding (10⁻³ M [^{nat}Lu]RM2) and expressed
as mean ± SD (n = 6).

| GRPR ligand | Specific internalization (mean ± SD, n = 6) |
|---|---|
| [D-3-[¹²⁵I]I-Tyr⁶]MJ9 | 3.57 ± 0.57 |
| [¹⁷⁷Lu]RM2 | 2.92 ± 0.20 |
| [¹⁷⁷Lu]DOTA-[Hse⁷]MJ9 | 1.40 ± 0.16 |
| [¹⁷⁷Lu]DOTA-[Bta⁸]MJ9 | 2.26 ± 0.18 |
| [¹⁷⁷Lu]AMTG | 3.03 ± 0.18 |
| [¹⁷⁷Lu]AMTG2 | 5.88 ± 0.33 |
| [¹⁷⁷Lu]NeoBOMB1 | 13.91 ± 0.64 |

### Plasma Studies

In vitro stability of the synthesized GRPR ligands was determined in human plasma (FIGS. 1 to 4) whereas the stabilized ligand [¹⁷⁷Lu]AMTG as well as the reference [¹⁷⁷Lu]RM2 were further analyzed in murine plasma. Therefore, only 100 μL of murine plasma was added to the tracer solution (final volume of 200 μL) immediately after labeling was finished. According to Linder et al. (Bioconjugate Chem. 20, 1171-1178 (2009)), metabolism is more rapid in murine than in human plasma for which reason the experiment was terminated after incubation at 37° C. for 6±0.5 h (FIGS. 5 and 6). Nevertheless, due to a smaller volume the murine mixture was additionally examined after incubation at 37° C. for 72±2 h (FIGS. 7 and 8).

Comparing all four ¹⁷⁷Lu-labeled GRPR ligands in human plasma after incubation at 37° C. for 72±2 h (FIGS. 1 to 4), the amount of intact tracer showed significant differences. The in vitro stability of the reference ligand [¹⁷⁷Lu]RM2 (FIG. 1) was determined to only 33.5±2.7% after that timespan whereas the stabilities of [¹⁷⁷Lu]DOTA-[Hse⁷]MJ9 (40.1±1.4%) as well as [¹⁷⁷Lu]AMTG (77.6±10.1%) were higher after incubation at 37° C. for 72±2 h. The most lipophilic derivative [¹⁷⁷Lu]DOTA-[Bta⁸]MJ9 (19.0±1.7%) showed the least stability of these four compounds. A second reference ligand, [¹⁷⁷Lu]NeoBOMB1 exhibited an amount of intact tracer of 60.8±1.2% after the same timespan.

The reference compound [¹⁷⁷Lu]RM2 (FIGS. 5 and 7) as well as the stabilized derivative [¹⁷⁷Lu]AMTG (FIGS. 6 and 8) were further examined in murine plasma to determine possible differences between human and animal plasma. According to Linder et al., metabolism in murine plasma after about 6 h is comparable to its counterpart in human plasma after about 3 d. Therefore, the stability of these two ligands was determined after 6 h in murine plasma revealing a comparable amount of intact [¹⁷⁷Lu]AMTG (t_{R}=17.0 min, 89%, FIGS. 6 and 92%, FIG. 4, respectively), but a significant deviation of intact ligand for the [¹⁷⁷Lu]RM2 ((R=15.5 min, 92%, FIG. 5 and 36%, FIG. 1, respectively).

Examination of these two tracers in murine plasma after a longer timespan (incubation at 37° C. for 72±2 h) showed that [¹⁷⁷Lu]RM2 (FIG. 7) is cleaved by murine endopeptidases at more sites than [¹⁷⁷Lu]AMTG (FIG. 8) but nevertheless, the amount of intact tracer seemed to be higher (67% and 59%, respectively) which led to the assumption that there are major differences between human and animal plasma, especially for the reference compound.

Considering these observations, the stabilized [¹⁷⁷Lu]AMTG shows a superior performance than the reference ligand in vivo in humans but not necessarily in mice.

### Biodistribution and μSPECT/CT Studies

In vivo pharmacokinetics of the reference compounds [¹⁷⁷Lu]RM2 as well as the diagnostic ligands [¹⁷⁷Lu]DOTA-[Hse⁷]MJ9 and [¹⁷⁷Lu]DOTA-[Bta⁸]MJ9 were examined in CB17-SCID mice at 1 h p.i. and 24 h p.i., whereas the therapeutic ligands [¹⁷⁷Lu]AMTG, [¹⁷⁷Lu]AMTG2 and a second reference [¹⁷⁷Lu]NeoBOMB1 were only studied at 24 h p.i. (100 pmol each). Data is compared to the reference shown in FIGS. 9 to 12.

Both destabilized compounds show a superior pharmacokinetic profile as compared to the reference ligand in mice at 1 h p.i. (FIGS. 9 and 10). For every organ, uptake of diagnostic ligands is equal or lower than the reference, especially regarding the GRPR-positive pancreas which highlights a faster washout from this organ presumably caused by higher metabolism at the destabilized position. Interestingly, tumor uptake of both diagnostic ligands was superior than the reference compound (FIG. 9) which led to the assumption that tumor enrichment is possible to a higher level as could be achieved by [¹⁷⁷Lu]RM2. Further, as metabolism in the tumor is less rapid than in non-tumor organs, there were no negative washout effects from the tumor at 1 h p.i. despite the destabilized bonds in the diagnostic derivatives.

As illustrated by the tumor-to-background ratios (FIG. 10), [¹⁷⁷Lu]DOTA-[Hse⁷]MJ9 showed the highest contrast between tumor and non-tumor organs at 1 h p.i. whereas the reference showed the worst of these three.

For a possible application for therapeutical purposes, the three ligands as well as [¹⁷⁷Lu]NeoBOMB1 and the stabilized [¹⁷⁷Lu]AMTG and [¹⁷⁷Lu]AMTG2 were investigated in CB17-SCID mice at 24 h p.i. (FIGS. 11 and 12). The pharmacokinetic profiles confirm the suggestion of a faster washout of the destabilized ligands from the tumor after a longer timespan. While retention of all four compared ligands was similar in normal tissues there was a significant difference in tumor retention as there were still high amounts of the [¹⁷⁷Lu]RM2, [¹⁷⁷Lu]AMTG and [¹⁷⁷Lu]AMTG2 in the tumor but only small amounts of the destabilized compounds. [¹⁷⁷Lu]NeoBOMB1 also revealed high tumor retention but high pancreas retention as well. Bone uptake for all derivatives can be explained by [¹⁷⁷Lu]LuCl₃ which was not fully complexed by the respective chelator (FIG. 11).

[¹⁷⁷Lu]RM2, [¹⁷⁷Lu]AMTG and [¹⁷⁷Lu]AMTG2 revealed a higher tumor retention than the other derivatives of this series at 24 h p.i. (FIG. 11). Considering the tumor-to-background ratios after that timespan, [¹⁷⁷Lu]AMTG and [¹⁷⁷Lu]AMTG2 exhibited superior tumor-to-blood as well as tumor-to-muscle ratios (FIG. 12). Both destabilized ligands as well as [¹⁷⁷Lu]NeoBOMB1 showed minor tumor-to-background ratios than the reference. Imaging studies with [¹⁷⁷Lu]RM2 and [¹⁷⁷Lu]AMTG at 1, 4, 8, 24 and 28 h p.i. on PC-3 tumor-bearing mice (100 pmol each) indicate the distribution in vivo over time (FIG. 19). Both conjugates exhibited favorable pharmacokinetics, each showing fast clearance from GRPR-positive tissues (pancreas, intestine) and high retention in the tumor. Background activity was cleared less rapid in case of [¹⁷⁷Lu]AMTG, especially from the pancreas, which was expected due to increased metabolic stability in vivo.

In conclusion, considering the results, both destabilized ligands outperformed the reference in mice at 1 h p.i., but were noticeably inferior in mice at 24 h p.i. which correlated well for [¹⁷⁷Lu]DOTA-[Bta⁸]MJ9 with the observations in the plasma studies as this compound showed the least metabolic stability in vitro. As mentioned before, metabolism in non-tumor tissue is faster than in tumor tissue leading to the well-known washout effects of GRPR antagonists. Further destabilization of the Gln⁷-Trp⁸ bond led to a more rapid washout from the background but not from the tumor which yielded in a superior contrast compared to the reference at 1 h p.i. Though, after a longer timespan a significantly faster washout from the tumor could be observed which confirmed the assumption of an increased enzymatic degradation of the more lipophilic [¹⁷⁷Lu]DOTA-[Bta⁸]MJ9. For this reason, it could be a useful diagnostic agent.

The other destabilized derivative [¹⁷⁷Lu]DOTA-[Hse⁷]MJ9 did not show a minor in vitro stability in human plasma but its in vivo behavior in mice demonstrated a minor stability as it demonstrated a faster clearance from non-tumor tissues at 1 h p.i. and a minor retention in the tumor at 24 h p.i. which confirms the suggestion of a minor metabolic stability.

The stabilized compound [¹⁷⁷Lu]AMTG showed particularly good overall performance considering the results in vitro and in vivo. It possesses good affinity to GRPR-expressing PC-3 cells, a reasonable lipophilicity, the highest metabolic stability in human plasma and equal or enhanced pharmacokinetic properties compared to [¹⁷⁷Lu]RM2. Due to its enhanced metabolic stability in vitro in human plasma, AMTG might have the potential to compete with or even outperform the current golden standards among GRPR-targeted ligands (RM2, NeoBOMB1) for targeted radiotherapy of GRPR-expressing malignancies in men.

### Example 7: [^{99m}Tc]N₄-Containing Ligands

### Examined Compounds

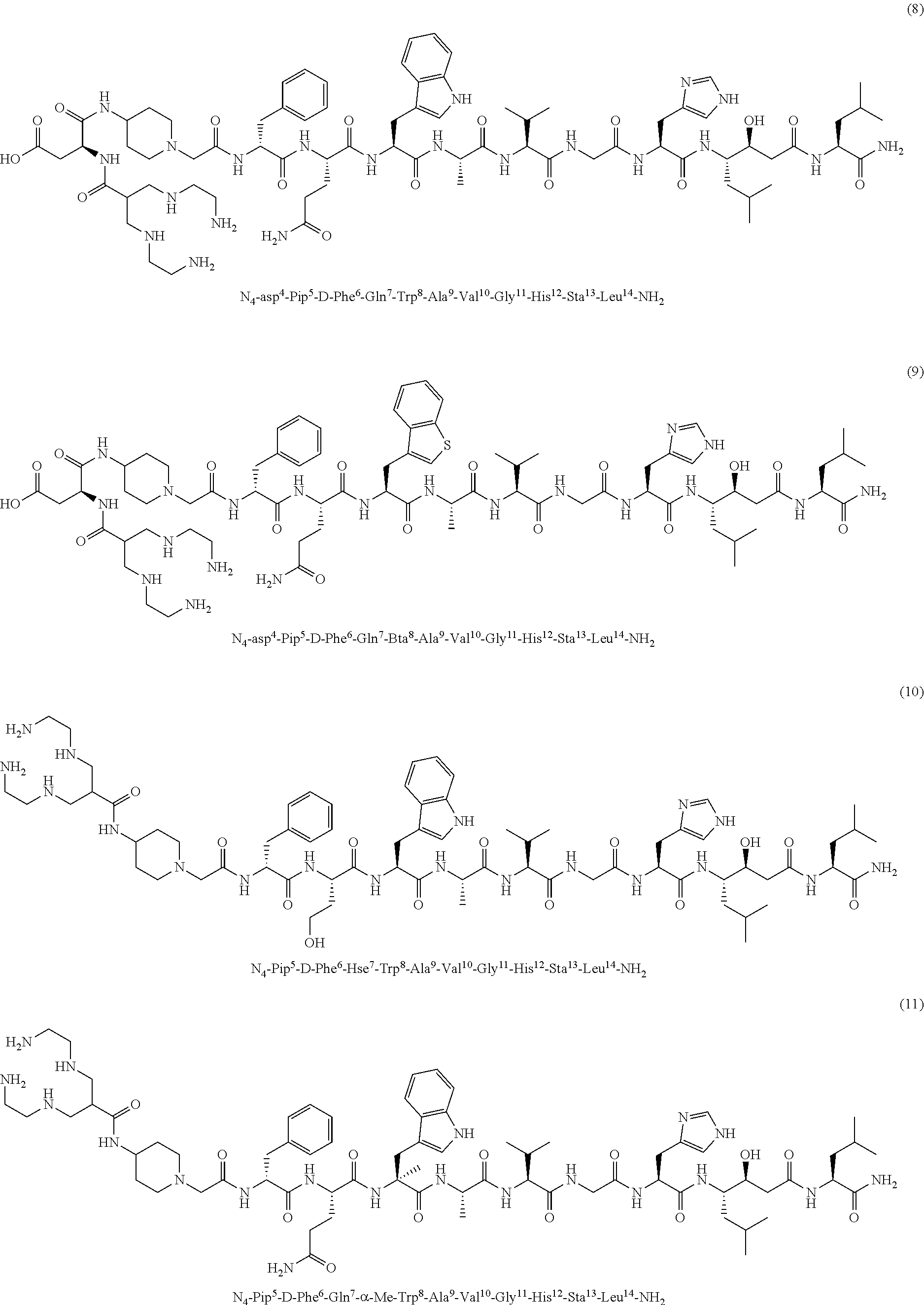

### In Vitro Data

The determined n-octanol/PBS distribution coefficients (log D_{7.4}) as well as the binding affinities (IC₅₀) towards GRPR of the ^{99m}Tc-labeled compounds are presented in Table 5. For all compounds, N4 (6-(carboxy))-1,4,4,11-tetraazaundecane) was used as a chelator.

TABLE 5
Distribution coefficients (logD_{7.4} values) as well as binding affinities
(IC₅₀) towards GRPR of [^{99m}Tc]N₄-asp-MJ9 (8), [^{99m}Tc]N₄-asp-
[Bta⁸]MJ9 (9), [^{99m}Tc]N₄-[Hse⁷]MJ9 (10) and [^{99m}Tc]N₄-
[α-Me-Trp⁸]MJ9 (11). Binding affinities were determined on
PC-3 cells (1.5 × 10⁵ cells/well) and [D-3-[¹²⁵I]I-Tyr⁶]MJ9
(c = 0.2 nM) as the radiolabeled reference (2 h, r.t., HBSS +
1% BSA). MJ9: Pip⁵-D-Phe⁶-Gln⁷-α-Me-Trp⁸-Ala⁹-
Val¹⁰-Gly¹¹-His¹²-Sta¹³-Leu¹⁴-NH₂.

| | log D_{7.4} (n = 6), ^{99m}Tc- | |
|---|---|---|
| Ligand | labeled | IC₅₀ [nM] ± SD (n = 3) |
| N₄-asp-MJ9 | −1.86 ± 0.03 | 3.73 ± 0.78 |
| N₄-asp-[Bta⁸]MJ9 | −1.33 ± 0.06 | 3.36 ± 0.42 |
| N₄-[Hse⁷]MJ9 | −1.26 ± 0.02 | 6.97 ± 1.84 |
| N₄-[α-Me-Trp⁸]MJ9 | −1.29 ± 0.08 | 5.38 ± 0.47 |

Within this series, [^{99m}Tc]N₄-asp-MJ9 was found to be the most hydrophilic whereas the other three compounds revealed similar, but more lipophilic values. All conjugates exhibited IC₅₀ values (non-labeled) in a comparable low nanomolar range. Nevertheless, the homoserine and the α-methyl tryptophan derivative showed slightly decreased GRPR affinities compared to the other two ligands of this series.

### In Vivo Data

In vivo pharmacokinetics of the ^{99m}Tc-labeled ligands (8), (9), (10) and (11) were investigated at 1 and 4 h p.i. (200 pmol each) on CB17-SCID mice. [^{99m}Tc]N₄-asp-MJ9 (8), [^{99m}Tc]N₄-asp-[Bta⁸]MJ9 and [^{99m}Tc]N₄-[Hse⁷]MJ9 demonstrated excellent pharmacokinetics at 1 h p.i. with high tumor and low overall background accumulation (FIG. 13). [^{99m}Tc]N₄-asp-[Bta⁸]MJ9 showed the lowest uptake in the GRPR-positive pancreas which highlights the faster washout from this organ due to a higher metabolic rate at the destabilized position. [^{99m}Tc]N₄-[Hse⁷]MJ9 revealed the highest tumor and second lowest pancreas uptake of this series. [^{99m}Tc]N₄-[α-Me-Trp⁸]MJ9 revealed the highest pancreas accumulation, which is presumably caused by an enhanced metabolic stability because of the α-methyl tryptophan modification, as already described in former sections. Tumor-to-background ratios at 1 h p.i. were mostly in favor of [^{99m}Tc]N₄-asp-MJ9 consequently to its enhanced hydrophilic character due to the additional aspartate modification (FIG. 14). However, it is assumed that tumor-to-background ratios would be improved in case of [¹⁷⁷Lu]DOTA-asp-[Bta⁸]MJ9 and [¹⁷⁷Lu]DOTA-[Hse⁷]MJ9 if both compounds did show similar hydrophilicities as [^{99m}Tc]N₄-asp-MJ9.

Biodistribution studies at 4 h p.i. highlighted the temporal course of these ^{99m}Tc-labeled ligands in vivo (FIG. 15). Whereas [^{99m}Tc]N₄-[α-Me-Trp⁸]MJ9 revealed enhanced tumor accumulation compared to 1 h p.i., all other derivatives of this series showed decreased tumor values. This further strengthens the suggestion of an increased metabolic stability due to the α-methyl tryptophan modification. In case of [^{99m}Tc]technetium, this is not desired, as for diagnostic reasons, a high tumor uptake at 1 h p.i. (and not only at 4 h p.i.) as well as a faster clearance from background organs is desired. Nevertheless, this modification is of high use for therapeutic compounds, for example for ¹⁷⁷Lu-labeled ligands described above. As a faster clearance from background organs is beneficial for diagnostic compounds, [^{99m}Tc]N₄-asp-[Bta⁸]MJ9 is of ideal use because of its enhanced metabolic instability, which is highlighted by the uptake values in most organs at 4 h p.i. Tumor-to-background ratios at 4 h p.i. are shown in FIG. 16, revealing the highest for [^{99m}Tc]N₄-asp-MJ9 and [^{99m}Tc]N₄-[Hse⁷]MJ9 for most organs.

The superior contrast at 1 h p.i. enabled by the destabilizing modifications homoserine and 3-benzothienyl alanine were additionally highlighted by μSPECT/CT imaging (FIG. 17). Both, [^{99m}Tc]N₄-asp-[Bta⁸]MJ9 and [^{99m}Tc]N₄-[Hse⁷]MJ9 revealed a slightly enhanced contrast compared to the non-modified [^{99m}Tc]N₄-asp-MJ9 despite their increased lipophilic character. As expected, [^{99m}Tc]N₄-[α-Me-Trp⁸]MJ9 exhibited an inferior contrast to the other three derivatives, as the increased metabolic stability led to an decelerated pancreas and intestine clearance, which is not favorable for diagnosis.

### Example 8: Bombesin-SiFA Derivatives

### Examined Compounds

MJ9: H₂N-Leu-Sta-His-Gly-Val-Ala-Trp-Gln-D-Phe-Pip-

| | | | | | | | | |
|---|---|---|---|---|---|---|---|---|
| [Lu]GT50 | MJ9 | dap | orn | asp | asp | dap([Lu]DOTAGA) | dap(SiFA) | eue |
| [Lu]GT51 | MJ9 | dap | asp | orn | dap([Lu]DOTAGA) | dap(SiFA) | eue | |
| [Lu]GT52 | MJ9 | Pip | Pip | asp | dap([Lu]DOTAGA) | dap(SiFA) | eue | |
| [Lu]GT53 | MJ9 | Pip | asp | dap([Lu]DOTAGA) | dap(SiFA) | eue | | |

### In Vitro Data

The determined n-octanol/PBS distribution coefficients (log D_{7.4}) as well as the binding affinities (IC₅₀) towards GRPR of the Bombesin-SiFA compounds are presented in Table 6. For all compounds, DOTAGA was used as a chelator.

TABLE 6
Distribution coefficients (logD_{7.4} values) as well as binding
affinities (IC₅₀) towards GRPR of ^{177/nat}Lu-labeled GT50, GT51,
GT52 and GT53. Binding affinities were determined on PC-3 cells (1.5 ×
10⁵ cells/well) and [D-3-[¹²⁵I]I-Tyr⁶]MJ9 (c = 0.2
nM) as the radiolabeled reference (2 h, r.t., HBSS + 1% BSA).

| Ligand | log D_{7.4} (n = 6), ¹⁷⁷Lu-labeled | IC₅₀ [nM], ^{nat}Lu-labeled (n = 3) |
|---|---|---|
| GT50 | −1.96 ± 0.15 | 9.7 ± 1.0 |
| GT51 | −1.96 ± 0.15 | 14.3 ± 1.5 |
| GT52 | −2.05 ± 0.08 | 9.7 ± 2.1 |
| GT53 | −1.81 ± 0.15 | 19.6 ± 4.6 |

All four compounds of this series revealed similar hydrophilicities. IC₅₀ values were in a comparable range for [¹⁷⁷Lu]GT50 and [¹⁷⁷Lu]GT52 and slightly increased for [¹⁷⁷Lu]GT51 and [¹⁷⁷Lu]GT53.

### Biodistribution Studies

All four compounds comprise a SiFA moiety for ¹⁸F-labeling as well as a chelator for ⁶⁸Ga- or ¹⁷⁷Lu-labeling. This is a useful feature, because radiohybrid-based ligands offer ideal theranostic pairs, as they are chemically indistinguishable, irrespective whether [¹⁸F][^{nat}Ga/^{nat}Lu]ligand or [¹⁹F][⁶⁸Ga/¹⁷⁷Lu]ligand is applied. Biodistribution of the ¹⁷⁷Lu-labeled ligands GT50, GT51, GT52 and GT53 were evaluated at 24 h p.i. (100 pmol each) on CB17-SCID mice. All derivatives demonstrated low overall background retention, except for the liver and the kidneys (FIG. 18). Tumor retention was decreased compared to [¹⁷⁷Lu]RM2, [¹⁷⁷Lu]AMTG and [¹⁷⁷Lu]AMTG2 (FIG. 11). All Bombesin-SiFA conjugates have to be optimized, especially considering the high kidney and slightly enhanced liver retention. However, the evaluated ligands in this series proved the functionality of the radiohybrid-based concept.

1. A compound binding to an endogenous receptor, said compound comprising
(i) an oligopeptide comprising a dipeptide with Trp being the C-terminal amino acid of said dipeptide, wherein said Trp is replaced with an α-amino acid Xaa₂, whereby the stability in serum or plasma of the peptide bond connecting Xaa₂ to the N-terminally adjacent amino acid is increased as compared to the peptide bond connecting Trp to the N-terminally adjacent amino acid in an otherwise identical compound; and
(ii) a moiety capable of generating therapeutically effective radiation, said moiety being covalently bound to said oligopeptide.

2. The compound of claim 1, wherein said N-terminally adjacent amino acid in said dipeptide is L-Gln, D-Gln, L-His, D-His or Gly, preferably L-Gln.

3. The compound of claim 1, wherein said endogenous receptor is a peptide receptor overexpressed in cancer disease, such as Neuromedin-B receptor (Bombesin-1 receptor, NMBR), Gastrin-releasing peptide receptor (Bombesin-2 receptor, GRPR), Bombesin receptor subtype 3 (BRS-3) or Cholecystokinin-2 receptor (CCK-2R), and wherein preferably
(a) said binding is with a K_{D} of less or equal 15 nM; and/or
(b) said compound is a GRPR antagonist, preferably with an IC₅₀ of less or equal 15 nM.

4. A compound of formula (I) 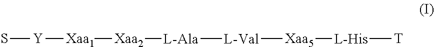
wherein
S is a moiety capable of generating therapeutically active radiation;
Y is an optional linker;
Xaa₁ is (i) L-Gln, D-Gln, L-His, D-His or Gly, preferably L-Gln; or
(ii) an α-amino acid which increases stability in serum or plasma of the Xaa₁-Xaa₂ peptide bond as compared to Xaa₁ being Gln and Xaa₂ being Trp in an otherwise identical compound;
Xaa₂ is Trp or an α-amino acid which increases stability in serum or plasma of the Xaa₁-Xaa₂ peptide bond as compared to Xaa₁ being Gln and Xaa₂ being Trp in an otherwise identical compound;
provided that Xaa₁ is not any one of L-Gln, D-Gln, L-His, D-His and Gly, and Xaa₂ is not Trp, respectively, at the same time;
Xaa₅ is Gly, N-Me-Gly, D-Ala, b-Ala or 2-aminoisobutyric acid (Aib); preferably Gly; and
T is an optional terminal group.

5. The compound of claim 1, wherein Xaa₂ is
(a) Trp which is modified to comprise
(i) a C1 to C4 optionally substituted alkyl moiety bound to the α-carbon, substituents being selected from halogen and hydroxyl; and/or
(ii) a substituent bound to the indole ring, substituents being selected from N-(2,2,2-trifluoromethyl), N-methyl, N-acetyl, 5-fluoro, 5-bromo, 5-iodo, 5-chloro, 5-hydroxy, 5-methoxy, 5-methyl, 6-chloro, 7-chloro and 7-Aza;
(b) 1,2,3,4-tetrahydro norharmane-3-carboxylic acid (L-Tpi).

6. The compound of claim 5, wherein said optionally substituted alkyl moiety is selected from —CH₃, CH₂CH₃, and CHₙHal₃₋ₙ, wherein n is 0, 1 or 2 and Hal is F, Cl, Br and/or I such as —CF₃; and preferably is —CH₃.

7. The compound of claim 1, wherein Xaa₂ is α-Me-Trp.

8. A compound of formula (II) 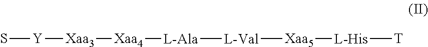
wherein
S is a moiety capable of generating a detectable signal;
Y is an optional linker;
Xaa₃ is (i) L-Gln, D-Gln, L-His, D-His or Gly, preferably L-Gln; or
(ii) an α-amino acid which decreases stability in serum or plasma of the Xaa₃-Xaa₄ peptide bond as compared Xaa₃ being Gln and Xaa₄ being Trp in an otherwise identical compound;
Xaa₄ is Trp or an α-amino acid which decreases stability in serum or plasma of the Xaa₃-Xaa₄ peptide bond as compared Xaa₃ being Gln and Xaa₄ being Trp in an otherwise identical compound;
wherein said α-amino acid at position Xaa₄ which decreases stability in serum or plasma of the Xaa₃-Xaa₄ peptide bond is not a proteinogenic amino acid;
provided that Xaa₃ is not any one of L-Gln, D-Gln, L-His, D-His and Gly, and Xaa₄ is not Trp, respectively, at the same time;
Xaa₅ is Gly, N-Me-Gly, b-Ala or 2-aminoisobutyric acid (Aib); preferably Gly; and
T is an optional terminal group.

9. The compound of claim 8, wherein Xaa₃ is Hse and/or Xaa₄ is Bta.

10. The compound of claim 4, wherein S is selected from a radioactive moiety and a moiety capable of being loaded with a radioactive nuclide.

11. The compound of claim 8, wherein S is selected from a fluorescent moiety, a radioactive moiety, and a moiety capable of being loaded with a radioactive nuclide.

12. The compound of claim 4, wherein Y is present and
(a) comprises one, two, three, four, five or six positive and/or negative charge(s);
(b) comprises or consists of one, two, three, four, five or six amino acids, preferably (a) D-amino acid(s) being among said amino acids, more preferably (a) D-α-amino acid(s);
(c) comprises or consists of PEGₙ, n being an integer selected from 1, 2, 3, 4, 5, 6, 7, 8, 9 and 10; and/or
(d) comprises a moiety capable of generating a detectable signal;
wherein preferably said linker Y comprises or consists of
(i) D-Glu-urea-D-Glu;
(ii) one or two 2,3-diaminopropionic acid moieties, optionally substituted with a moiety capable of generating a detectable signal;
(iii) one, two, three, four, five or six consecutive amino acids comprising or consisting of one or more amino acids selected from D-/L-aspartate, D-/L-ornithine, 4-amino-1-carboxymethyl-piperidine (Pip), D-/L-2,3-diaminopropionic acid, D-/L-serine, D-/L-citrulline moieties, L-cysteic acid (Ala(SO₃H)), amino-valeric acid (Ava), 4-aminobenzoic acid (PABA) and D-Phe; and/or
(iv) p-aminomethylaniline-diglycolic acid (pABza-DIG, AMA-DGA), and/or diglycolate (DIG, DGA).

13. The compound of claim 4, wherein T is present and comprises or consists of
(a) statine (Sta or (3S,4S)-4-amino-3-hydroxy-6-methylheptanoic acid), 2,6-dimethyl heptane, Leu or β-thienyl-L-alanine (Thi);
(b) Leu, norleucine (Nle), Pro, Met, or 1-amino-1-isobutyl-3-methyl-butane, wherein the amidic amine group of said Leu may be modified with ethyl (NH-Ethyl) or NH₂ (NH—NH₂); and/or
(c) (S)-1-((S)-2-amino-4-methylpentyl) pyrrolidine-2-carboxamide (Leu-ψ(CH₂N)-Pro-NH₂);
provided that if T is or terminates with an amino acid, the carboxylate of said amino acid is amidated.

14. A pharmaceutical composition comprising or consisting of a compound of claim 1.

15. A diagnostic composition comprising or consisting of a compound of claim 8.

16. The compound of claim 4, wherein Xaa₂ is
(a) Trp which is modified to comprise
(i) a C1 to C4 optionally substituted alkyl moiety bound to the α-carbon, substituents being selected from halogen and hydroxyl; and/or
(ii) a substituent bound to the indole ring, substituents being selected from N-(2,2,2-trifluoromethyl), N-methyl, N-acetyl, 5-fluoro, 5-bromo, 5-iodo, 5-chloro, 5-hydroxy, 5-methoxy, 5-methyl, 6-chloro, 7-chloro and 7-Aza;
(b) 1,2,3,4-tetrahydro norharmane-3-carboxylic acid (L-Tpi).

17. The compound of claim 4, wherein Xaa₂ is α-Me-Trp.

18. The compound of claim 8, wherein Y is present and
(a) comprises one, two, three, four, five or six positive and/or negative charge(s);
(b) comprises or consists of one, two, three, four, five or six amino acids, preferably (a) D-amino acid(s) being among said amino acids, more preferably (a) D-α-amino acid(s);
(c) comprises or consists of PEGₙ, n being an integer selected from 1, 2, 3, 4, 5, 6, 7, 8, 9 and 10; and/or
(d) comprises a moiety capable of generating a detectable signal;
wherein preferably said linker Y comprises or consists of
(i) D-Glu-urea-D-Glu;
(ii) one or two 2,3-diaminopropionic acid moieties, optionally substituted with a moiety capable of generating a detectable signal;
(iii) one, two, three, four, five or six consecutive amino acids comprising or consisting of one or more amino acids selected from D-/L-aspartate, D-/L-ornithine, 4-amino-1-carboxymethyl-piperidine (Pip), D-/L-2,3-diaminopropionic acid, D-/L-serine, D-/L-citrulline moieties, L-cysteic acid (Ala(SO₃H)), amino-valeric acid (Ava), 4-aminobenzoic acid (PABA) and D-Phe; and/or
(iv) p-aminomethylaniline-diglycolic acid (pABza-DIG, AMA-DGA), and/or diglycolate (DIG, DGA).

19. The compound of claim 8, wherein T is present and comprises or consists of
(a) statine (Sta or (3S,4S)-4-amino-3-hydroxy-6-methylheptanoic acid), 2,6-dimethyl heptane, Leu or β-thienyl-L-alanine (Thi);
(b) Leu, norleucine (Nle), Pro, Met, or 1-amino-1-isobutyl-3-methyl-butane, wherein the amidic amine group of said Leu may be modified with ethyl (NH-Ethyl) or NH₂ (NH—NH₂); and/or
(c) (S)-1-((S)-2-amino-4-methylpentyl) pyrrolidine-2-carboxamide (Leu-ψ(CH₂N)-Pro-NH₂);
provided that if T is or terminates with an amino acid, the carboxylate of said amino acid is amidated.

20. A pharmaceutical composition comprising or consisting of a compound of claim 4.
